(19) United States
(12) Patent Application Publication
(10) Pub. No.: US 2017/0226495 A1
(43) Pub. Date: Aug. 10, 2017

(54) SORTASE MOLECULES AND USES THEREOF

(71) Applicants: Carla Guimaraes, Cambridge, MA (US); Novartis AG, Basel (CH)

(72) Inventor: Carla Guimaraes, Cambridge, MA (US)

(21) Appl. No.: 15/327,816

(22) PCT Filed: Jul. 21, 2015
(86) PCT No.: PCT/US2015/041293

§ 371 Date: Jan. 20, 2017

Related U.S. Application Data
(60) Provisional application No. 62/027,137, filed on Jul. 21, 2014.

(51) Int. Cl. C12N 9/52

(52) U.S. Cl. CPC C12N 9/52; C12Y 304/2207

(57) ABSTRACT

This application provides mutant sortase molecules and methods of making and using them. In a first aspect, disclosed herein, are sortase molecules having one or a combination of mutations. In an embodiment, a sortase molecule is optimized for a parameter of enzyme performance, e.g., Ca++ dependency (or independency) or reaction rate.

This application claims priority to U.S. Ser. No. 62/027,137 filed Jul. 21, 2014, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 15, 2015, is named N2067-7056WO_SL.txt and is 31,208 bytes in size.

### FIELD OF THE INVENTION

The invention relates to sortase molecules and methods of making and using them.

### BACKGROUND OF THE INVENTION

Sortases are a family of enzymes that, in nature, play a role in the formation of the bacterial cell wall by covalently linking specific surface proteins to the peptidoglycan. Sortase enzymes carry out a transpeptidation reaction. In the first step of the reaction, the sortase cleaves a peptide bond in a sortase recognition motif, e.g., the peptide bond between a threonine and glycine/alanine residues in the sortase recognition motif, forming an acyl intermediate. In the second step, the sortase binds to an acceptor protein bearing a sortase acceptor motif, e.g., several N-terminal glycine residues, and transfers the acyl intermediate to the N-terminus of the sortase acceptor motif. The end result is formation of a new peptide bond between the C-terminus of the protein and the N-terminus of the precursor of the cell wall component.

### SUMMARY

Disclosed herein are mutant sortase molecules. These molecules can be used to covalently couple, by way of sortase molecule mediated transfer, a moiety coupled to a sortase recognition motif to a moiety coupled to a sortase acceptor motif. By way of example, a sortase molecule disclosed herein can be used to couple a moiety, e.g., a target binding moiety, to another moiety, e.g., a polypeptide or cell, rapidly and under physiological conditions.

In a first aspect, disclosed herein, are sortase molecules having one or a combination of mutations. In an embodiment, a sortase molecule is optimized for a parameter of enzyme performance, e.g., Ca++ dependency (or independency) or reaction rate.

In one embodiment, the sortase molecule comprises the amino acid sequence of SEQ ID NO:3, comprising: a mutation selected from Pro94 (P94), Asp160 (D160), Asp165 (D165), Lys190 (K190), and Lys196 (K196); a mutation selected from Glu105 (E105) and Glu108 (E108); and having at least 80, 85, 90, or 95% homology with SEQ ID NO:3. (Residue numbering is with reference to the full length wild-type sequence, provided in SEQ ID NO:1 herein.)

In one embodiment, the sortase molecule comprises the amino acid sequence of SEQ ID NO:3, comprising: a mutation selected from Pro94 (P94), Asp160 (D160), Asp165 (D165), Lys190 (K190), and Lys196 (K196); a mutation selected from Glu105 (E105) and Glu108 (E108); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising: a mutation selected from: Pro94 (P94), Asp160 (D160), Asp165 (D165), Lys190 (K190), and Lys196 (K196); and a mutation selected from Glu105 (E105) and Glu108 (E108).

In one embodiment, the sortase molecule comprises a fragment of the amino acid sequence of SEQ ID NO:3, comprising: a mutation selected from Pro94 (P94), Asp160 (D160), Asp165 (D165), Lys190 (K190), and Lys196 (K196); and a mutation selected from Glu105 (E105) and Glu108 (E108), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E), and Lys196Thr (K196T); a mutation selected from Glu105Lys (E105K) and Glu108Gln (E108Q); and having at least 80, 85, 90, or 95% homology with SEQ ID NO:3.

In one embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E), and Lys196Thr (K196T); and a mutation selected from Glu105Lys (E105K) and Glu108Gln (E108Q); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q).

In one embodiment, the sortase molecule comprises a fragment of the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising: a mutation selected from Pro94 (P94), Asp160 (D160), Asp165 (D165), Lys190 (K190) and Lys196 (K196); and 1 or 2 mutations selected from Glu105 (E105) and Glu108 (E108); and having at least 80, 85, 90, or 95% homology with SEQ ID NO:3.

In one embodiment, the sortase molecule comprises the amino acid sequence of SEQ ID NO:3, comprising: a mutation selected from Pro94 (P94), Asp160 (D160), Asp165 (D165), Lys190 (K190) and Lys196 (K196); and 1 or 2 mutations selected from Glu105 (E105) and Glu108 (E108); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising: a mutation selected from Pro94 (P94), Asp160 (D160), Asp165 (D165), Lys190 (K190) and Lys196 (K196); and 1 or 2 mutations selected from Glu105 (E105) and Glu108 (E108).

In one embodiment, the sortase molecule comprises a fragment of the amino acid sequence of SEQ ID NO 3, comprising: a mutation selected from Pro94 (P94), Asp160 (D160), Asp165 (D165), Lys190 (K190) and Lys196 (K196); and 1 or 2 mutations selected from Glu105 (E105) and Glu108 (E108), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q).

In one embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); 1 or 2 mutations selected from Glu105Lys (E105K) and Glu108Gln (E108Q); and having at least 80, 85, 90, or 95% homology with SEQ ID NO:3.

In one embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q); and otherwise differing from SEQ ID NO:3 by no more than 1, 2, 3, 4, 5, 6, 7, 8, 9, or 10 amino acid residues.

In one embodiment, the sortase molecule comprises a fragment of the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising: 2, 3, 4, or 5 mutations selected from Pro94 (P94), Asp160 (D160), Asp165 (D165), Lys190 (K190) and Lys196 (K196); and 1 or 2 mutations selected from Glu105 (E105) and Glu108 (E108); and having at least 80, 85, 90, or 95% homology with SEQ ID NO:3.

In one embodiment, the sortase molecule comprises the amino acid sequence of SEQ ID NO:3, comprising: 2, 3, 4, or 5 mutations selected from Pro94 (P94), Asp160 (D160), Asp165 (D165), Lys190 (K190) and Lys196 (K196); and 1 or 2 mutations selected from Glu105 (E105) and Glu108 (E108), and otherwise differing from SEQ ID NO:3 by no more than 1,2, 3, 4, 5, 6, 7, 8, 9, or 10 amino acid residues.

In one embodiment, the sortase molecule comprises the amino acid sequence of SEQ ID NO:3, comprising: 2, 3, 4, or 5 mutations selected from Pro94 (P94), Asp160 (D160), Asp165 (D165), Lys190 (K190) and Lys196 (K196); and 1 or 2 mutations selected from Glu105 (E105) and Glu108 (E108).

In one embodiment, the sortase molecule comprises a fragment of the amino acid sequence of SEQ ID NO:3, comprising: 2, 3, 4, or 5 mutations selected from Pro94 (P94), Asp160 (D160), Asp165 (D165), Lys190 (K190) and Lys196 (K196); and 1 or 2 mutations selected from Glu105 (E105) and Glu108 (E108), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising: 2, 3, 4, or 5 mutations selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q); and having at least 80, 85, 90, or 95% homology with SEQ ID NO:3.

In one embodiment, the sortase molecule comprises the amino acid sequence of SEQ ID NO:3, comprising: 2, 3, 4, or 5 mutations selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising: 2, 3, 4, or 5 mutations selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q).

In one embodiment, the sortase molecule comprises a fragment of the amino acid sequence of SEQ ID NO:3, comprising: 2, 3, 4, or 5 mutations selected from Pro94Arg (P94R), Asp160Asn (D160N), Asp165Ala (D165A), Lys190Glu (K190E) and Lys196Thr (K196T); and 1 or 2 mutations selected from Glu105Lys (E105K) and Glu108Gln (E108Q), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising the following mutations, Pro94 (P94), Glu105 (E105), Glu108 (E108), Asp160 (D160), Asp165 (D165), Lys190 (K190), and Lys196 (K196); and having at least 80, 85, 90, or 95% homology with SEQ ID NO:3.

In one embodiment, the sortase molecule comprises the amino acid sequence of SEQ ID NO:3, comprising the following mutations, Pro94 (P94), Glu105 (E105), Glu108 (E108), Asp160 (D160), Asp165 (D165), Lys190 (K190), and Lys196 (K196) and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising the following mutations: Pro94 (P94), Glu105 (E105), Glu108 (E108), Asp160 (D160), Asp165 (D165), Lys190 (K190), and Lys196 (K196).

In one embodiment, the sortase molecule comprises a fragment of the amino acid sequence of SEQ ID NO:3, comprising the following mutations: Pro94 (P94), Glu105 (E105), Glu108 (E108), Asp160 (D160), Asp165 (D165), Lys190 (K190), and Lys196 (K196), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising the following mutations, Pro94Arg (P94R), Glu105Lys (E105K), Glu108Gln (E108Q), Asp160Asn (D160N), Asp165Ala (D165A), Lys190Glu (K190E), and Lys196Thr (K196T); and having at least 80, 85, 90, or 95% homology with SEQ ID NO:3.

In one embodiment, the sortase molecule comprises the amino acid sequence of SEQ ID NO:3, comprising the following mutations, Pro94Arg (P94R), Glu105Lys (E105K), Glu108Gln (E108Q), Asp160Asn (D160N), Asp165Ala (D165A), Lys190Glu (K190E), and Lys196Thr (K196T); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising the following mutations, Pro94Arg (P94R), Glu105Lys (E105K), Glu108Gln (E108Q), Asp160Asn (D160N), Asp165Ala (D165A), Lys190Glu (K190E), and Lys196Thr (K196T).

In one embodiment, the sortase molecule comprises a fragment of the amino acid sequence of SEQ ID NO:3, comprising the following mutations, Pro94Arg (P94R), Glu105Lys (E105K), Glu108Gln (E108Q), Asp160Asn (D160N), Asp165Ala (D165A), Lys190Glu (K190E), and Lys196Thr (K196T), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, a mutation at any of the following Pro94 (P94), Asp160 (D160), Asp165 (D165), Lys190 (K190) and Lys196 (K196); and having at least 80, 85, 90, or 95% homology with SEQ ID NO:3.

In one embodiment, the sortase molecule comprises the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, a mutation at any of the following Pro94 (P94), Asp160 (D160), Asp165 (D165), Lys190 (K190) and Lys196 (K196); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, a mutation at any of the following Pro94 (P94), Asp160 (D160), Asp165 (D165), Lys190 (K190) and Lys196 (K196).

In one embodiment, the sortase molecule comprises a fragment of the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, a mutation at any of the following Pro94 (P94), Asp160 (D160), Asp165 (D165), Lys190 (K190) and Lys196 (K196), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, the sortase molecule comprises the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, any of the following Pro94Arg (P94R), Asp160Asn (D160N), Asp165Ala (D165A), Lys190Glu (K190E) and Lys196Thr (K196T); and having at least 80, 85, 90, or 95% homology with SEQ ID NO:3.

In one embodiment, the sortase molecule comprises the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, any of the following Pro94Arg (P94R), Asp160Asn (D160N), Asp165Ala (D165A), Lys190Glu (K190E) and Lys196Thr (K196T), and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, any of the following Pro94Arg (P94R), Asp160Asn (D160N), Asp165Ala (D165A), Lys190Glu (K190E) and Lys196Thr (K196T).

In one embodiment, the sortase molecule comprises a fragment of the amino acid sequence of SEQ ID NO:3, comprising an uncharged replacement, e.g., an uncharged amino acid selected from Ala, Ser, Thr, Asn, Gln, Trp, Phe, Pro, Gly, Met, Leu, Val, Ile, Cys, Tyr, and His or a positively charged replacement, e.g., a positively charged amino acid is selected from Lys and Arg, at one or both of Glu105 (E105) and Glu108 (E108), and optionally, any of the following Pro94Arg (P94R), Asp160Asn (D160N), Asp165Ala (D165A), Lys190Glu (K190E) and Lys196Thr (K196T), wherein the fragment is capable of transferring a moiety attached to a sortase recognition motif to a moiety comprising a sortase acceptor motif. In an embodiment the fragment is at least 100, 105, 110, 115, 120, 125, 130, 135, 140, or 145 amino acid residues in length.

In one embodiment, Glu105 (E105) is mutated to an uncharged or positively charged amino acid. In one embodiment, Glu108 (E108) is mutated to an uncharged or positively charged amino acid. In one embodiment, an uncharged amino acid is selected from Ala, Ser, Thr, Asn, Gln, Trp, Phe, Pro, Gly, Met, Leu, Val, Ile, Cys, Tyr, and His. In one embodiment, a positively charged amino acid is selected from Lys and Arg.

In an embodiment, a sortase molecule comprises an amino acid sequence that is homologous, e.g., 60, 70, 80, 85, 90, 95, or 99% homologous, to a sortase amino acid sequence described herein, and the sortase molecule retains the desired functional properties of the sortase described herein, e.g., the ability to transfer a moiety attached to a sortase recognition motif to a moiety comprising a sortase acceptor motif.

In one embodiment, the sortase molecule comprises the amino acid sequence of SEQ ID NO:3, comprising a mutation selected from the following, Pro94 (P94), Glu105 (E105), Glu108 (E108), Asp160 (D160), Asp165 (D165), Lys190 (K190) and Lys196 (K196); and having at least 80, 85, 90, or 95% homology with SEQ ID NO:3.

In one embodiment, the sortase molecule comprises the amino acid sequence of SEQ ID NO:3, comprising a mutation selected from the following, Pro94 (P94), Glu105 (E105), Glu108 (E108), Asp160 (D160), Asp165 (D165), Lys190 (K190) and Lys196 (K196); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising a mutation selected from the following: Pro94 (P94), Glu105 (E105), Glu108 (E108), Asp160 (D160), Asp165 (D165), Lys190 (K190) and Lys196 (K196).

In one embodiment, the sortase molecule comprises the amino acid sequence of SEQ ID NO:3, comprising a mutation selected from the following: Pro94Arg (P94R), Glu105Lys (E105K), Glu108Gln (E108Q), Asp160Asn (D160N), Asp165Ala (D165A), Lys190Glu (K190E) and Lys196Thr (K196T); and having at least 80, 85, 90, or 95% homology with SEQ ID NO:3.

In one embodiment, the sortase molecule comprises the amino acid sequence of SEQ ID NO:3, comprising a mutation selected from the following: Pro94Arg (P94R), Glu105Lys (E105K), Glu108Gln (E108Q), Asp160Asn (D160N), Asp165Ala (D165A), Lys190Glu (K190E) and Lys196Thr (K196T); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising a mutation selected from the following: Pro94Arg (P94R), Glu105Lys (E105K), Glu108Gln (E108Q), Asp160Asn (D160N), Asp165Ala (D165A), Lys190Glu (K190E) and Lys196Thr (K196T).

In one embodiment, the sortase molecule comprises the amino acid sequence of SEQ ID NO:3, comprising 2, 3, 4, 5, 6, or 7 mutations selected from the following: Pro94 (P94), Glu105 (E105), Glu108 (E108), Asp160 (D160), Asp165 (D165), Lys190 (K190) and Lys196 (K196); and having at least 80, 85, 90, or 95% homology with SEQ ID NO:3.

In one embodiment, the sortase molecule comprises the amino acid sequence of SEQ ID NO:3, comprising 2, 3, 4, 5, 6, or 7 mutations selected from the following: Pro94 (P94), Glu105 (E105), Glu108 (E108), Asp160 (D160), Asp165 (D165), Lys190 (K190) and Lys196 (K196); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising 2, 3, 4, 5, 6, or 7 mutations selected from the following: Pro94 (P94), Glu105 (E105), Glu108 (E108), Asp160 (D160), Asp165 (D165), Lys190 (K190) and Lys196 (K196).

In one embodiment, the sortase molecule comprises the amino acid sequence of SEQ ID NO:3, comprising 2, 3, 4, 5, 6, or 7 mutations selected from the following: Pro94Arg (P94R), Glu105Lys (E105K), Glu108Gln (E108Q), Asp160Asn (D160N), Asp165Ala (D165A), Lys190Glu (K190E) and Lys196Thr (K196T); and having at least 80, 85, 90, or 95% homology with SEQ ID NO:3.

In one embodiment, the sortase molecule comprises the amino acid sequence of SEQ ID NO:3, comprising 2, 3, 4, 5, 6, or 7 mutations selected from the following: Pro94Arg (P94R), Glu105Lys (E105K), Glu108Gln (E108Q), Asp160Asn (D160N), Asp165Ala (D165A), Lys190Glu (K190E) and Lys196Thr (K196T); and otherwise differing from SEQ ID NO:3 by no more than 1, 2, 3, 4, 5, 6, 7, 8, 9, or 10 amino acid residues.

In one embodiment, the sortase molecule comprises the amino acid sequence of SEQ ID NO:3, comprising 2, 3, 4, 5, 6, or 7 mutations selected from the following: Pro94Arg (P94R), Glu105Lys (E105K), Glu108Gln (E108Q), Asp160Asn (D160N), Asp165Ala (D165A), Lys190Glu (K190E) and Lys196Thr (K196T).

In an embodiment, a sortase molecule described herein does not comprise additional sortase sequence N terminal to SEQ ID NO:3.

In an embodiment, a sortase molecule described herein comprises additional sequence, e.g., sortase sequence, N terminal to the N terminus of SEQ ID NO:3.

In an embodiment a sortase molecule comprises, e.g., at its N terminal end 1, 2, 3, 4, 5, 6, 10, 20, 30, 40, 50, or 59 consecutive amino acid residues from SEQ ID NO: 2.

In an embodiment a sortase molecule comprises, e.g., at its N terminal end, a methionine.

In an embodiment a sortase molecule comprises, e.g., at its N terminal end, less than 1, 2, 3, 4, 5, 6, 10, 20, 30, 40, 50, or 59 consecutive amino acid residues from SEQ ID NO: 2.

In an embodiment, a sortase molecule described herein does not comprise additional sortase sequence C terminal to SEQ ID NO:3.

In an embodiment a sortase molecule comprises, e.g., at its C terminal end, additional sequence, e.g., a sequence tag useful for purification, e.g., a His tag, e.g., a 3×HIS tag, a 6×HIS tag (SEQ ID NO: 32), or an 8×HIS tag (SEQ ID NO: 33).

In some embodiments, the sortase molecule is a purified or isolated preparation.

In a second aspect, disclosed herein, is a nucleic acid, e.g., a DNA, e.g., a cDNA, or RNA, or a purified or isolated preparation thereof, that encodes a sortase molecule described herein.

In a third aspect, disclosed herein, is a vector comprising a nucleic acid, e.g., a DNA, e.g., a cDNA, or RNA, that encodes a sortase molecule described herein.

In a fourth aspect, disclosed herein, is a cell, e.g., a prokaryotic cell, e.g., an E. coli cell, comprising a nucleic acid or vector that comprises sequence that encodes a sortase molecule described herein.

In a fifth aspect, disclosed herein, is a method of making a sortase molecule, comprising, providing a cell, e.g., a prokaryotic cell, e.g., an E. coli cell, comprising a nucleic acid or vector that comprises sequence that encodes a sortase molecule, and recovering a sortase molecule from the cell or secreted by the cell.

In a sixth aspect, disclosed herein, is a method of making a complex comprising a sortase molecule and a cleaved sortase recognition motif, comprising:

contacting a sortase recognition motif with a sortase molecule, e.g., under conditions that allow for the formation of the complex, e.g., under conditions allowing for cleavage of the sortase recognition motif and coupling to the sortase molecule,

thereby making a complex comprising the sortase molecule and a cleaved sortase recognition motif,

provided that, the sortase molecule is a sortase molecule of any of claims 1-10.

In an embodiment, the cleaved sortase recognition motif is coupled to a moiety. In an embodiment, the moiety comprises a polypeptide. In an embodiment, the moiety comprises a marker. In an embodiment, the moiety comprises a target binding molecule. In an embodiment, the moiety comprises an antibody molecule. In an embodiment, the sortase recognition motif comprises LPXTA/G, wherein X is any amino acid.

In a seventh aspect, disclosed herein, is a complex comprising a sortase molecule described herein and a cleaved sortase recognition motif. In an embodiment, the cleaved sortase recognition motif is coupled to a moiety. In an embodiment, the moiety comprises a polypeptide. In an embodiment, the moiety comprises a marker. In an embodiment, the moiety comprises a target binding molecule. In an embodiment, the moiety comprises an antibody molecule. In an embodiment, the cleaved sortase recognition motif comprises at least X residues from LPXT wherein X is equal to 1, 2, 3, or 4.

In an eighth aspect, disclosed herein, is a method of coupling a first moiety to a second moiety, comprising:
a) providing the first moiety coupled to a sortase acceptor motif and the second moiety coupled to a sortase recognition motif:
b) contacting the first moiety coupled to a sortase acceptor motif with:
(i) a sortase molecule and the second moiety coupled to a sortase recognition motif; or
(ii) a complex comprising the second moiety coupled to a cleaved sortase recognition motif and a sortase molecule;

under conditions sufficient to allow transfer of a second moiety coupled to a cleaved sortase recognition motif to the sortase acceptor motif coupled to the first moiety,

thereby coupling a first moiety to a second moiety, provided that, the sortase molecule is a sortase molecule described herein.

In an embodiment, the first moiety comprises a polypeptide. In an embodiment, the first moiety comprises a marker. In an embodiment, the first moiety comprises a target binding molecule. In an embodiment, the first moiety comprises an antibody molecule.

In an embodiment, the method of coupling a first moiety to a second moiety comprises contacting the first moiety coupled to a sortase acceptor motif with a sortase molecule and the second moiety coupled to a sortase recognition motif.

In an embodiment, the method of coupling a first moiety to a second moiety comprises contacting the first moiety coupled to a sortase acceptor motif with a complex comprising the second moiety coupled to a cleaved sortase recognition motif and a sortase molecule.

In an embodiment, the sortase molecule comprises the amino acid sequence of SEQ ID NO:3, comprising: a mutation selected from Pro94 (P94), Asp160 (D160), Asp165 (D165), Lys190 (K190) and Lys196 (K196); and a mutation selected from Glu105 (E105) and Glu108 (E108); and otherwise differing from SEQ ID NO:3 by no more than 1 2, 3, 4, 5, 6, 7, 8, 9, or 10 amino acid residues.

In an embodiment, the sortase molecule comprises the amino acid sequence of SEQ ID NO:3, comprising: a mutation selected from Pro94 (P94), Asp160 (D160), Asp165 (D165), Lys190 (K190) and Lys196 (K196); and a mutation selected from Glu105 (E105) and Glu108 (E108).

In an embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q).

In an embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q), and having at least 90% homology with SEQ ID NO:3.

In an embodiment, the sortase molecule comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q); and otherwise differing from SEQ ID NO:3 by no more than 1 2, 3, 4, 5, 6, 7, 8, 9, or 10 amino acid residues.

In an embodiment, the sortase molecule comprises the amino acid sequence of SEQ ID NO:3, comprising the following mutations, Pro94 (P94), Glu105 (E105), Glu108 (E108), Asp160 (D160), Asp165 (D165), Lys190 (K190) and Lys196 (K196) and having at least 90% homology with SEQ ID NO:1.

In an embodiment, the sortase molecule comprises the amino acid sequence of SEQ ID NO:3, comprising the following mutations, Pro94 (P94), Glu105 (E105), Glu108 (E108), Asp160 (D160), Asp165 (D165), Lys190 (K190) and Lys196 (K196); and otherwise differing from SEQ ID NO:3 by no more than 1 2, 3, 4, 5, 6, 7, 8, 9, or 10 amino acid residues.

In an embodiment, the sortase molecule comprises the amino acid sequence of SEQ ID NO:3, comprising the following mutations: Pro94 (P94), Glu105 (E105), Glu108 (E108), Asp160 (D160), Asp165 (D165), Lys190 (K190), and Lys196 (K196).

In an embodiment, the sortase molecule comprises the amino acid sequence of SEQ ID NO: 5.

In an embodiment, the first moiety comprises a polypeptide.

In an embodiment, the second moiety comprises a polypeptide. In an embodiment, the second moiety comprises a marker. In an embodiment, the second moiety comprises a target binding molecule. In an embodiment, the second moiety comprises an antibody molecule.

In an embodiment, the first moiety comprises a first polypeptide and the second moiety comprises a second polypeptide. In an embodiment, the first polypeptide and the second polypeptide have the same structure, e.g., the same primary amino acid sequence. In an embodiment, the first polypeptide and the second polypeptide differ in structure, e.g., they have different primary amino acid sequences.

In an embodiment, the first or second polypeptide is a transmembrane polypeptide. In an embodiment, the first polypeptide is a transmembrane polypeptide, e.g., having an extracellular domain comprising a sortase acceptor motif. In an embodiment, the first or second polypeptide comprises the extracellular domain of a transmembrane polypeptide. In an embodiment, the second polypeptide comprises the extracellular domain of a transmembrane polypeptide.

In an embodiment, the first or second polypeptide comprises an antibody molecule or a target binding molecule. In an embodiment, the second polypeptide comprises an antibody molecule or a target binding molecule.

In an embodiment, the first or second polypeptide is disposed in a cell, e.g., a transmembrane polypeptide. In an embodiment, the first or second polypeptide is disposed in a cell, e.g., a transmembrane polypeptide disposed in the cell membrane. In an embodiment, the first polypeptide is disposed in a cell, e.g., a transmembrane polypeptide disposed in the cell membrane.

In an embodiment, the first polypeptide is disposed in or on a cell, e.g., as a transmembrane polypeptide, and the method comprises contacting the cell with:
(i) a sortase molecule and the second moiety coupled to a sortase recognition motif; or
(ii) a complex comprising the second moiety coupled to a cleaved sortase recognition motif and a sortase molecule.

In an embodiment, the method of coupling a first moiety to a second moiety comprises contacting the cell with a sortase molecule and the second moiety coupled to a sortase recognition motif.

In an embodiment, the method of coupling a first moiety to a second moiety comprises contacting the cell with a complex comprising the second moiety coupled to a cleaved sortase recognition motif and a sortase molecule.

In an embodiment, the second polypeptide is disposed in or on a cell, e.g., as a transmembrane polypeptide which is coupled to:

(i) a sortase recognition motif; or

(ii) a complex comprising a cleaved sortase recognition motif and a sortase molecule. In an embodiment, the method of coupling a first moiety to a second moiety further comprises contacting the cell with first moiety coupled to a sortase acceptor motif.

In an embodiment, the method of coupling a first moiety to a second moiety further comprises contacting the cell with first moiety coupled to a sortase acceptor motif and a sortase.

In an embodiment, the sortase acceptor motif comprises an amino acid residue, e.g., a Gly or Ala residue, which accepts transfer of a moiety by the sortase.

In an embodiment, the sortase acceptor motif comprises an amino acid residue, e.g., a Gly or Ala residue, which accepts transfer of a moiety mediated by nucleophilic attack. In an embodiment, the sortase acceptor motif comprises, consists of, or consists essentially of, Gly-, Gly-Gly-, Gly-Gly-Gly-, Gly-Gly-Gly-Gly- (SEQ ID NO: 34), or Gly-Gly-Gly-Gly-Gly- (SEQ ID NO: 35). In an embodiment, the sortase acceptor motif comprises, Gly-, Gly-Gly-, Gly-Gly-Gly-, Gly-Gly-Gly-Gly- (SEQ ID NO: 34), or Gly-Gly-Gly-Gly-Gly-(SEQ ID NO: 35). In an embodiment, the sortase acceptor motif comprises, consists of, or consists essentially of, Ala-, Ala-Ala-, Ala-Ala-Ala-, Ala-Ala-Ala-Ala-(SEQ ID NO: 36), or Ala-Ala-Ala-Ala-Ala- (SEQ ID NO: 37). In an embodiment, the sortase acceptor motif comprises, Ala-, Ala-Ala-, Ala-Ala-Ala-, Ala-Ala-Ala-Ala-(SEQ ID NO: 36), or Ala-Ala-Ala-Ala-Ala- (SEQ ID NO: 37).

In a ninth aspect, disclosed herein, is a method of providing a cell having a moiety attached thereto, comprising

a) providing a sortase acceptor motif coupled to a first moiety, e.g., a precursor cell or a first moiety disposed in or on a precursor cell;

b) contacting the precursor cell with
(i) a sortase molecule and a second moiety coupled to a sortase recognition motif; or
(ii) a complex comprising the second moiety coupled to a cleaved sortase recognition motif and a sortase molecule.
   under conditions sufficient to allow transfer of a second moiety coupled to a cleaved sortase recognition motif to the sortase acceptor motif coupled to the first moiety, provided that, the sortase molecule is a sortase molecule described herein, thereby providing cell having a moiety attached thereto.

In an embodiment, the method of providing a cell having a moiety attached thereto comprises:

c) contacting the precursor cell with
(i) a sortase molecule and a third moiety coupled to a sortase recognition motif; or
(ii) a complex comprising the third moiety coupled to a cleaved sortase recognition motif and a sortase molecule;
   under conditions sufficient to allow transfer of a third moiety coupled to a cleaved sortase recognition motif to the sortase acceptor motif coupled to the first moiety, thereby providing a cell having a second and a third moiety attached thereto.

In an embodiment, step b and c are performed simultaneously.

In an embodiment, the structures of the second and third moieties are different.

In an embodiment, the second moiety comprises a target binding molecule. In an embodiment, the second moiety comprises a target binding molecule and the third moiety comprises a target binding molecule.

In an embodiment, the second moiety comprises binding target binding molecule and the third moiety comprises a target binding molecule, and they bind the same target. In an embodiment, the second moiety and the third moiety bind the same target with different affinities. In an embodiment, the second moiety and the third moiety bind different targets.

In an embodiment, the second moiety or the third moiety comprises a marker, e.g., a luciferase, dye, or fluorophore. In an embodiment, the second moiety and the third moiety each comprises a marker, e.g., a luciferase, dye, or fluorophore.

In a tenth aspect, disclosed herein, is a reaction mixture comprising a sortase molecule described herein. In an embodiment, the reaction mixture further comprises a sortase recognition motif. In an embodiment, the reaction mixture further comprises a sortase acceptor motif. In an embodiment, the reaction mixture further comprises a precursor cell comprising a sortase acceptor motif. In an embodiment, the reaction mixture further comprises a first moiety coupled to a sortase acceptor motif.

In an embodiment, the reaction mixture further comprises a second moiety coupled to a sortase recognition motif and a third moiety coupled to a sortase recognition motif. In an embodiment, the structures of the second and third moieties are different. In an embodiment, the second moiety comprises a target binding molecule. In an embodiment, the second moiety and the third moiety comprises a target binding molecule. In an embodiment, the second moiety and the third moiety comprises a target binding molecule and bind to the same target. In an embodiment, the second moiety and the third moiety bind the same target with different affinities. In an embodiment, the second moiety and the third moiety bind different targets.

In an embodiment, the second moiety or the third moiety comprises a marker, e.g., a dye, fluorophore, or radionuclide. In an embodiment, the second moiety and the third moiety comprises a marker, e.g., a dye, fluorophore, or radionuclide.

In an eleventh aspect, disclosed herein, is a reaction mixture comprising:

a complex comprising a cleaved sortase recognition motif, and any sortase molecule described herein.

In an embodiment, the reaction mixture further comprises a sortase acceptor motif. In an embodiment, the reaction mixture further comprises a precursor cell comprising a sortase acceptor motif.

In a twelfth aspect, disclosed herein, is a reaction mixture comprising a first sortase molecule and a second sortase molecule, wherein the first sortase molecule is a sortase molecule described herein, and/or the second sortase molecule is a sortase molecule described herein.

In an embodiment, the first sortase molecule and the second sortase molecule are different.

In an embodiment, the first sortase molecule is a sortase molecule described herein, e.g., a mutant sortase molecule, and the second sortase molecule is a wild-type sortase molecule, e.g., from S. aureus, S. pyogenes, Actionomyces naeslundii, Bacillus anthracis, Bacillus cereus, Bacillus halodurans, Bacillus subtilis, Bifidobacterium longum, Clostridium botunlinum, Clostridium difficile, Corynebacterium diphtheriae, Corynebacterium efficiens, Corynebacterium glutamicum, Enterococcus faecium, Geobacillus sp. Listeria innocua, Listeria monocytogenes, Oceanobacillus iheyensis, Ruminococcus albus, Streptomyces avermitilis, Streptomyces coelicolor, Streptomyces griseus, Staphylococcus epidermis, Streptococcus agalactiae, Streptococcus equi, Streptococcus gordonii, Streptococcus pyogenes, Thermobifida fusca, Tropheryma wipplei.

In an embodiment, the reaction mixture further comprises a first moiety coupled to a first sortase acceptor motif, a second moiety coupled to a second sortase acceptor motif, a third moiety coupled to a first sortase recognition motif, and a fourth moiety coupled to a second sortase recognition motif.

In an embodiment, the first moiety and the second moiety are the same, and wherein the third moiety and the fourth moiety are the same.

In an embodiment, the first moiety and the second moiety are different, and wherein the third moiety and the fourth moiety are the same.

In an embodiment, the first moiety and the second moiety are different, and wherein the third moiety and the fourth moiety are different.

In an embodiment, the third moiety and/or the fourth moiety is a target binding molecule.

In an embodiment, the third moiety and/or the fourth moiety is a marker, e.g., a luciferase, a dye, a fluorophore.

In a thirteenth aspect, disclosed herein, is a method of providing a purified preparation of a first moiety coupled to a second moiety, comprising:

providing the first moiety coupled to the second moiety, e.g., comprising a sortase transfer signature, and

separating the first moiety coupled to the second moiety from a sortase molecule,

thereby providing a purified preparation of a first moiety coupled to a second moiety,

wherein the sortase molecule is any sortase molecule described herein.

In an embodiment, the method of providing a purified preparation of a first moiety coupled to a second moiety, comprises
a) providing the first moiety coupled to a sortase acceptor motif and the second moiety coupled to a sortase recognition motif:
b) contacting the first moiety coupled to a sortase acceptor motif with:
(i) a sortase molecule and the second moiety coupled to a sortase recognition motif; or
(ii) a complex comprising the second moiety coupled to a cleaved sortase recognition motif and a sortase molecule;

under conditions sufficient to allow transfer of a second moiety coupled to a cleaved sortase recognition motif to the sortase acceptor motif coupled to the first moiety,

thereby coupling a first moiety to a second moiety, and

separating the sortase molecule from first moiety coupled to the second moiety, provided that, the sortase molecule is a sortase molecule described herein.

In a fourteenth aspect, disclosed herein, is a method of providing a first moiety coupled to a second moiety comprising:

providing a mixture comprising (i) first moiety coupled to a second moiety, and comprising, e.g., a sortase transfer signature; and (ii) a sortase molecule of described herein; and

separating the sortase from the cell, thereby providing a first moiety coupled to a second moiety.

In a fifteenth aspect, disclosed herein, is a first moiety coupled to a second moiety, made by the method of providing a first moiety coupled to a second moiety described herein.

In a sixteenth aspect, disclosed herein, is a method of providing a cell having a first conjugate and a second conjugate attached thereto, comprising
a) providing a first sortase acceptor motif coupled to a first moiety, e.g., coupled to a precursor cell or disposed in or on a precursor cell,
b) providing a second sortase acceptor motif coupled to a second moiety, e.g., coupled to a precursor cell or disposed in or on the precursor cell;
c) contacting the precursor cell with:
   (i) a first sortase molecule and a third moiety coupled to a first sortase recognition motif, or
   (ii) a complex comprising the third moiety coupled to a cleaved first sortase recognition motif and a second sortase molecule; and
d) contacting the precursor cells with:
   (iii) a second sortase molecule and a fourth moiety coupled to a second sortase recognition motif; or
   (iv) a complex comprising the fourth moiety coupled to a cleaved second sortase recognition motif and a second sortase molecule;
      under conditions sufficient to allow transfer of a third moiety coupled to a cleaved first sortase recognition motif to the first sortase acceptor motif coupled to the first moiety to generate a first conjugate, and transfer of a fourth moiety coupled to a cleaved second sortase recognition motif to the second sortase acceptor motif coupled to the second moiety to generate a second conjugate,

thereby providing the cell having a first conjugate and a second conjugate attached thereto, e.g., wherein the first conjugate comprises the first moiety and the third moiety, and the second conjugate comprises the second moiety and the fourth moiety.

In an embodiment, steps a) and b) are performed simultaneously.

In an embodiment, steps a) and c) are performed before steps b) and d).

In an embodiment, steps b) and d) are performed before steps a) and c).

In an embodiment, steps a), b), c) and c) are performed simultaneously.

In an embodiment, the first sortase molecule and the second sortase molecule are different.

In an embodiment, the first sortase molecule and the second sortase molecule are the same.

In an embodiment, the first sortase molecule and/or the second sortase molecule is any sortase molecule described herein.

In an embodiment, the first sortase molecule is any sortase molecule described herein, and the second sortase molecule is a wild-type sortase A, e.g., from S. aureus, S. pyogenes, Actionomyces naeslundii, Bacillus anthracis, Bacillus cereus, Bacillus halodurans, Bacillus subtilis, Bifidobacterium longum, Clostridium botunlinum, Clostridium difficile, Corynebacterium diphtheriae, Corynebacterium efficiens, Corynebacterium glutamicum, Enterococcus faecium, Geobacillus sp. Listeria innocua, Listeria monocytogenes, Oceanobacillus iheyensis, Ruminococcus albus, Streptomyces avermitilis, Streptomyces coelicolor, Streptomyces griseus, Staphylococcus epidermis, Streptococcus agalactiae, Streptococcus equi, Streptococcus gordonii, Streptococcus pyogenes, Thermobifida fusca, Tropheryma wipplei.

In an embodiment, the structures of the first moiety and the second moiety are the same.

In an embodiment, the structures of the first moiety and the second moiety are different.

In an embodiment, the structures of the third moiety and the fourth moiety are the same.

In an embodiment, the structures of the third moiety and the fourth moiety are different.

In an embodiment, the third moiety comprises a target binding molecule.

In an embodiment, the third moiety comprises a target binding molecule and the fourth moiety comprises a target binding molecule. In an embodiment, the third moiety and the fourth bind the same target. In an embodiment, the third moiety and the fourth moiety bind the same target with different affinities.

In an embodiment, the third moiety and the fourth moiety bind different targets.

In an embodiment, the third moiety or the fourth moiety comprises a marker, e.g., a luciferase, dye, or fluorophore. In an embodiment, the third moiety and the fourth moiety each comprises a marker, e.g., a luciferase, dye, or fluorophore.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic representation of C-terminal labeling of proteins. A protein modified at its C terminus with the LPXTG (SEQ ID NO: 38) sortase-recognition motif followed by a handle (e.g., His6 (SEQ ID NO: 32)) is incubated with S. aureus Sortase A. Sortase cleaves the threonine-glycine bond and via its active site cysteine residue forming an acyl intermediate with threonine in the protein. Addition of a peptide probe comprising a series of N-terminal glycine residues and a functional moiety of choice resolves the intermediate, thus regenerating the active site cysteine (HS) on sortase and ligating the peptide probe to the C terminus of the protein.

FIG. 2 is an image demonstrating labeling of a scFV directed to the CD19 protein harboring a LPXTG (SEQ ID NO: 38) sortase-recognition motif followed by a His8 (SEQ ID NO: 33) at its C-terminus (scFV19, 20 μM) with either WT (40 μM) or mutant [P94R/E105K/E108Q/D160N/D165A/K190E/K196T] sortase A (40 μM), in the presence or absence of 10 mM calcium chloride, and G₃K(TAMRA) peptide (SEQ ID NO: 7) (1 mM), at 37° C., for the times indicated. The reactions were analyzed by reducing SDS-PAGE followed by fluorescent scanning (bottom panel) and coomassie-blue staining (upper panel). The molecular weight markers are shown on the left. The predicted identity of the various protein bands observed in the gel is indicated by the arrows. The Figure discloses “LPETG” and “LPETG₃K” as SEQ ID NOS 39 and 49, respectively.

FIG. 3 is an image demonstrating labeling of a scFV directed to the CD19 protein harboring a LPXTG (SEQ ID NO: 38) sortase-recognition motif followed by a His8 (SEQ ID NO: 33) at its C-terminus (scFV19, 20 μM) with the mutant [P94R/E105K/E108Q/D160N/D165A/K190E/K196T] sortase A (40 μM), G₃K(TAMRA) peptide (SEQ ID NO: 7) (1 mM) in RPMI+1% FBS media supplemented or not with 50 mM Tris-Cl, pH 7.4, 150 mM NaCl buffer, at 37° C., for the times indicated. The reactions were monitored by reducing SDS-PAGE, followed by fluorescent scanning (bottom panel) and coomassie-blue staining (upper panel).

FIG. 4 is an image demonstrating labeling of a scFV directed to the CD19 protein harboring a LPXTG (SEQ ID NO: 38) sortase-recognition motif followed by a His8 (SEQ ID NO: 33) at its C-terminus (scFV19, 20 μM) with the mutant [P94R/E105K/E108Q/D160N/D165A/K190E/K196T] sortase A (40 μM or 120 μM), G₃K(TAMRA) peptide (SEQ ID NO: 7) (1 mM) in 50 mM Tris-Cl, pH 7.4, 150 mM NaCl buffer, at the temperatures and times indicated. The reactions were monitored by reducing SDS-PAGE, followed by fluorescent scanning and coomassie-blue staining. The molecular weight markers are shown on the left. The predicted identity of the various protein bands observed in the gel is indicated by the arrows. The Figure discloses “LPETG” and “LPETG₃K” as SEQ ID NOS 39 and 49, respectively.

FIG. 5 shows a graph of untransduced K562 cells or K562 cells expressing CD19 at their surface incubated for 30 min at 4° C. with various concentrations of a scFV directed to CD19 which had been conjugated to TAMRA (scFV19.LPETG-TAMRA_conjugated) (“LPETG” disclosed as SEQ ID NO: 39) through a sortase-mediated reaction. As a control, scFV19 subjected to the same reaction conditions to label the scFV with TAMRA, but omitting sortase (scFV19.LPETG+TAMRA_not conjugated) (“LPETG” disclosed as SEQ ID NO: 39) was used. Flow cytometry analysis comparing cell labeling is shown.

FIG. 6, comprising FIGS. 6A and 6B, is a series of schematic representations of the process for conjugating an apelin peptide to an Fc molecule by using Sortase A (FIG. 6A) and the process for preparing the apelin peptide containing a sortase acceptor motif for the sortase-mediated reaction (FIG. 6B).

FIG. 7, comprising FIGS. 7A and 7B, is a series of schematic representations of the process for conjugating another apelin peptide to an Fc molecule by using Sortase A (FIG. 7A) and the process for preparing the apelin peptide containing a sortase acceptor motif for the sortase-mediated reaction (FIG. 7B).

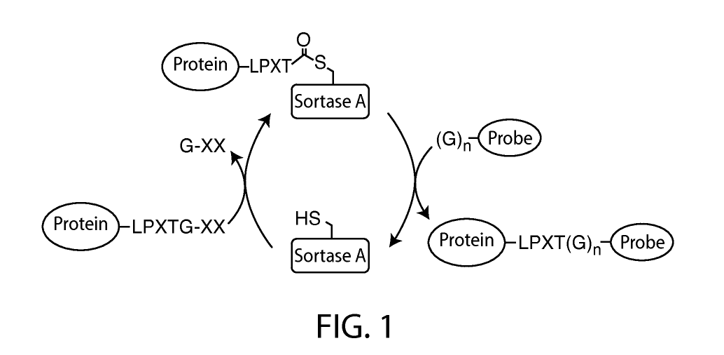

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an”, as used herein, refer to one or to more than one (e.g., to at least one) of the grammatical object of the article.

The term “or” as used herein, means, and is used interchangeably with, the term “and/or”, unless context clearly indicates otherwise.

The terms “about” and “approximately”, as used herein shall generally mean an acceptable degree of error for the quantity measured given the nature or precision of the measurements. Exemplary degrees of error are within 20 percent (%), typically, within 10%, and more typically, within 5% of a given value or range of values.

The term “antibody molecule”, as used herein, refers to an immunoglobulin, e.g., an antibody, and to antigen binding portions thereof, e.g., molecules that contain an antigen binding site which specifically binds an antigen, such as a polypeptide. A molecule which specifically binds to a given polypeptide, but does not substantially bind other molecules in a sample, e.g., a biological sample, which naturally contains the polypeptide. Antibody molecules include “antibody fragments” which refers to a portion of an intact antibody that is sufficient to confer recognition and specific binding to a target antigen. Examples of antibody fragments include, but are not limited to, Fab, Fab′, F(ab′)2, and Fv fragments, linear antibodies, scFv antibodies, a linear antibody, single domain antibody (sdAb), e.g., either a variable light (VL) chain or a variable heavy (VH) chain, a camelid VHH domain, and multispecific antibodies formed from antibody fragments. Antibody molecules can be polyclonal or monoclonal. The term “monoclonal” as applied to antibody molecules herein, refers to a population of antibody molecules that contain only one species of an antigen binding site capable of immunoreacting with a particular epitope.

The term “isolated” nucleic acid molecule, as used herein, is one which is separated from other nucleic acid molecules which are present in the natural, or synthetic, source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized. The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, less than about 20%, less than about 10%, or less than about 5% (by dry weight) of heterologous protein (also referred to herein as a “contaminating protein”). When the protein or biologically active portion thereof is recombinantly produced, it can be substantially free of culture medium, i.e., culture medium represents less than about 20%, less than about 10%, or less than about 5% of the volume of the protein preparation. When the protein is produced by chemical synthesis, it can substantially be free of chemical precursors or other chemicals, i.e., it is separated from chemical precursors or other chemicals which are involved in the synthesis of the protein. Accordingly such preparations of the protein have less than about 30%, less than about 20%, less than about 10%, less than about 5% (by dry weight) of chemical precursors or compounds other than the polypeptide of interest.

A “marker”, as used herein, refers to a molecule that can be used for identification, detection, purification, or isolation. In an embodiment, the marker comprises a small molecule, a peptide, a polypeptide, or a labeled amino acid or nucleotide. In an embodiment, the marker generates a signal for detection, e.g., a radioactive signal, a chemiluminescent signal, a fluorescent signal, or a chromogenic signal. For example, the marker is a dye, a fluorophore, a reporter enzyme (e.g., a photoprotein, luciferase), a fluorescent peptide, or a radionuclide. The generated signal can be detected by a variety of assays known in the art, such as fluorescence microscopy, fluorescence-activated cell sorting, gel electrophoresis, and spectrophotometry.

“A moiety” coupled to a sortase acceptor motif, as that term is used herein, refers to a molecule which is to be attached to a cleaved sortase recognition motif. In an embodiment the moiety comprises an amino acid, peptide, polypeptide, sugar, nucleic acid or other biological molecule. In an embodiment the moiety comprises a marker, or signal generating molecule, e.g., a dye, or radionuclide. The moiety can be coupled to a sortase acceptor motif covalently or non-covalently. In an embodiment the moiety and a sortase acceptor motif are a fusion polypeptide. In an embodiment the moiety comprises a transmembrane polypeptide.

“A moiety” coupled to a sortase recognition motif, as that term is used herein, refers to a molecule which is to be attached to a sortase acceptor motif. In an embodiment the moiety comprises an amino acid, peptide, polypeptide, sugar, nucleic acid or other biological molecule. In an embodiment the moiety comprises a marker, or signal generating molecule, e.g., a dye, or radionuclide. The moiety can be coupled to a sortase recognition motif covalently or non-covalently. In an embodiment the moiety and a sortase recognition motif are a fusion polypeptide. In an embodiment, the moiety comprises a target binding molecule. In an embodiment, the moiety comprises an antibody molecule. In an embodiment, the moiety comprises small molecules or ligands and/or counterligands that are on the surface of a cell, e.g., a cancer cell.

“Sortase,” as that term is used herein, refers to a molecule which catalyzes a transpeptidase reaction between a sortase recognition motif and a sortase acceptor motif. In an embodiment, the sortase molecule catalyzes a reaction to couple a first moiety to a second moiety by a peptide bond.

In an embodiment, sortase mediated transfer is used to couple the N terminus of a first polypeptide to the N terminus of a second polypeptide. In such embodiments, sortase mediated transfer is used to attach a coupling moiety, e.g., a “click” handle, to the N terminus of each polypeptide, e.g., the first polypeptide and the second polypeptide, wherein the coupling moieties mediate coupling of the polypeptides. In an embodiment the first polypeptide comprises a sortase acceptor motif, and the second polypeptide comprises a sortase acceptor motif. Sortase mediated transfer is used to attach a coupling moiety, e.g., a click handle, to each polypeptide, and a click chemistry reaction is used to couple the N terminus of the first polypeptide to the N terminus of the second polypeptide.

“Sortase acceptor motif,” as that term is used herein, refers to a moiety that acts as an acceptor for the sortase-mediated transfer of a polypeptide to the sortase acceptor motif. In an embodiment the sortase acceptor motif is located at the N terminus of a polypeptide. In an embodiment the transferred polypeptide is linked by a peptide bond at its C terminus to the N terminal residue of the sortase acceptor motif. N-terminal acceptor motifs include Gly-[Gly]ₙ- (SEQ ID NO: 40), wherein n=0-5 and Ala-[Ala]ₙ-(SEQ ID NO: 41), wherein n=0-5.

“Sortase recognition motif,” as that term is used herein, refers to a polypeptide which, upon cleavage by sortase molecule forms a thioester bond with the sortase molecule. In an embodiment, the sortase recognition motif comprises LPXTG/A, wherein X is any amino acid. In an embodiment, sortase cleavage occurs between T and G/A. In an embodiment the peptide bond between T and G/A is replaced with an ester bond to the sortase molecule.

“Sortase transfer signature,” as that term is used herein, refers to the portion of a sortase recognition motif and the portion of a sortase acceptor motif remaining after the reaction that couples the former to the latter. In an embodiment, wherein the sortase recognition motif is LPXTG/A and wherein the sortase acceptor motif is GG, the resultant sortase transfer signature after sortase-mediated reaction is LPXTGG (SEQ ID NO: 42).

A “target binding molecule” as the term is used herein, refers to a molecule that has affinity for a target molecule. A target binding molecule can comprise, e.g., a binding partner, e.g., a ligand or receptor, from a ligand-receptor system. A target binding molecule can comprise an antibody molecule, e.g., an antibody or antigen binding fragment thereof, single domain antibody (sdAb), or a single chain antibody (scFv). A target binding molecule can comprise a non-antibody scaffold, e.g., a fibronectin, or the like. In an embodiment, a sortase molecule is used to attach a target binding molecule to another moiety.

### Sortase Mutants

One aspect of the invention pertains to an isolated sortase molecule comprising a mutant sortase sequence. In one embodiment, a sortase molecule can be isolated from cells or tissue sources by an appropriate purification scheme using standard protein purification techniques. In another embodiment, a sortase molecule is produced by recombinant DNA techniques. In one embodiment a sortase molecule is produced in vivo, e.g., in an organism or in cultured cells. Alternative to recombinant expression, a sortase molecule can be synthesized chemically using standard peptide synthesis techniques.

The amino acid sequence of wild-type S. aureus sortase A, full length, (GenBank: BAB43619.1) is as follows:

(SEQ ID NO: 1)
MKKWTNRLMT IAGVVLILVA AYLFAKPHID NYLHDKDKDE
KIEQYDKNVK EQASKDNKQQ AKPQIPKDKS KVAGYIEIPD
ADIKEPVYPG PATPEQLNRG VSFAEENESL DDQNISIAGH
TFIDRPNYQF TNLKAAKKGS MVYFKVGNET RKYKMTSIRD
VKPTDVEVLD EQKGKDKQLT LITCDDYNEK TGVWEKRKIF
VATEVK

The N-terminal 59 amino acids of S. aureus sortase A (GenBank: BAB43619.1) is as follows:

(SEQ ID NO: 2)
MKKWTNRLMT IAGVVLILVA AYLFAKPHID NYLHDKDKDE
KIEQYDKNVK EQASKDNKQ

The amino acid sequence of wild-type S. aureus sortase A, starting at position 60 (having amino acids 1-59 truncated), is as follows:

(SEQ ID NO: 3)
QAKPQIPKD KSKVAGYIEI PDADIKEPVY PGPATPEQLN
RGVSFAEENE SLDDQNISIA GHTFIDRPNY QFTNLKAAKK
GSMVYFKVGN ETRKYKMTSI RDVKPTDVEV LDEQKGKDKQ
LTLITCDDYN EKTGVWEKRK IFVATEVK

The nucleotide sequence of wild-type S. aureus sortase A (GenBank: NC_002745.2) is provided below:

(SEQ ID NO: 4)
ATGAAAAAATGGACAAATCGATTAATGACAATCGCTGGTGTAGTACTTAT
CCTAGTGGCAGCATATTTGTTTGCTAAACCACATATCGATAATTATCTTC
ACGATAAAGATAAAGATGAAAAGATTGAACAATATGATAAAAATGTAAAA
GAACAGGCGAGTAAAGACAATAAGCAGCAAGCTAAACCTCAAATTCCGAA
AGATAAATCAAAAGTGGCAGGCTATATTGAAATTCCAGATGCTGATATTA
AAGAACCAGTATATCCAGGACCAGCAACACCTGAACAATTAAATAGAGGT
GTAAGCTTTGCAGAAGAAAATGAATCACTAGATGATCAAAATATTTCAAT
TGCAGGACACACTTTCATTGACCGTCCGAACTATCAATTTACAAATCTTA
AAGCAGCCAAAAAAGGTAGTATGGTGTACTTTAAAGTTGGTAATGAAACA
CGTAAGTATAAAATGACAAGTATAAGAGATGTTAAGCCAACAGATGTAGA
AGTTCTAGATGAACAAAAAGGTAAAGATAAACAATTAACATTAATTACTT
GTGATGATTACAATGAAAAGACAGGCGTTTGGGAAAAACGTAAAATCTTT
GTAGCTACAGAAGTCAAATAA

Methods described herein can be used to make and test additional candidate sortase mutants, starting, e.g., from wildtype or mutant sortase sequences provided herein.

Mutant sortase molecules can be optimized for one or more parameters, including the ability to operate under relatively mild conditions and to have a relatively high turnover, which can be important in reactions involving labile substrates or components. For example, when using a sortase molecule to attach a polypeptide or other moiety to another polypeptide or moiety, a living cell, or other labile substrate, it can be advantageous for the reaction to proceed without high concentrations of calcium and/or to proceed relatively quickly.

In an embodiment, a mutant sortase molecule described herein is optimized for one or more of the following parameters or conditions:

Reaction conditions: The sortase molecule is active under reaction conditions that are physiological or close to physiological, e.g., in terms of pH (i.e., neutral), temperature (25° C.−37° C.), and buffer conditions;

Kinetics: The sortase molecule should display fast kinetics to afford maximization of the amount of a given functional group, e.g., moiety, to be attached. In the case of attachment to a living cell, the kinetics should maximize the number of molecules attached to another moiety, polypeptide, or cell surface per round of sortase-mediated reaction.

Reliability: The sortase molecule should be reliable, with the sortase molecule accepting the moiety attached to the sortase recognition motif, e.g., a polypeptide, in active or native conformation, e.g., a correctly folded polypeptide, e.g., antibody. The sortase molecule should also reliably attach the moiety in the same spatially oriented manner (e.g., through the C-terminus, thus leaving the N-terminus available for antigen recognition).

Low interference and immunogenicity: The sequence resultant from the reaction of the sortase recognition motif and the sortase acceptor motif (e.g., the sortase transfer signature) should be minimal to avoid interfering with the activity of the product, e.g, a cell having a moiety, e.g., a polypeptide attached thereto by virtue of the sortase molecule, and to reduce the likelihood of an immunogenic response against this site.

Site-Specificity: The sortase molecule catalyzed reaction which transfers the moiety should be to a great extent site-specific to maximize the formation of the proper construct, e.g., upon attachment of a moiety, e.g., a polypeptide, to a cell.

Calcium dependence: Use of 10 mM calcium for S. aureus sortase A activity is not ideal in some uses, as high calcium can affect or interfere with biological processes. Thus, the sortase molecules described herein may have decreased dependence on calcium for activity or may be calcium independent.

An example of a mutant sortase molecule is Sortase A mutant [P94R/E105K/E108Q/D160N/D165A/K190E/K196T]. It lacks the N-terminal 59 amino acids of S. aureus sortase A and includes mutations that render the enzyme calcium independent and which make the enzyme faster. (The number of residues herein begin with residue the first residue at the N terminal end of non-truncated S. aureus Sortase A.). The primary amino acid sequence is provided below. Mutations are in bold. The underlined residue is E in this embodiment but can be any amino acid, e.g., a conservative substitution. The sequence of Sortase A mutant [P94R/E105K/E108Q/D160N/D165A/K190E/K196T] is as follows:

(SEQ ID NO: 5)
MQAKPQIPKD KSKVAGYIEI PDADIKEPVY PGPATREQLN
RGVSFAKENQ SLDDQNISIA GHTFIDRPNY QFTNLKAAKK
GSMVYFKVGN ETRKYKMTSI RNVKPTAVEV LDEQKGKDKQ
LTLITCDDYN EETGVWETRK IFVATEVKLE HHHHHH

The present invention further provides an additional candidate sortase molecule that can be constructed from a wild-type sortase molecule or a mutant sortase molecule described herein. In an embodiment, 1, 2, 3, 4, 5, 6, 7, 8. 9, 10, 15, 20, 25 or 30 mutations can be introduced to a wild-type sortase molecule to construct an additional candidate sortase molecule. The wild-type sortase molecule can be any sortase molecule naturally, e.g., endogenously, expressed in a bacteria, e.g., a gram-positive bacteria, e.g., S. aureus, S. pyogenes. In an embodiment, an additional 1, 2, 3, 4, 5, 6, 7, 8. 9, 10, 15, 20, 25 or 30 mutations can be introduced to a mutant sortase molecule described herein to construct an additional candidate sortase molecule. The mutation may be point mutation (e.g., a silent, missense, or nonsense mutation), an insertion mutation, or a deletion mutation. The additional mutations introduced to a wild-type or sortase molecule described herein can improve or optimize a parameter, e.g., reaction conditions, calcium dependency, or kinetics. Standard molecular biology techniques and recombinant DNA methods for introducing mutations, e.g., to a nucleic acid encoding a wild-type or sortase molecule described herein, are known in the art. For example, PCR-based mutagenesis or chemical site-directed mutagenesis can be used to introduce a mutation to a wild-type or sortase molecule described herein.

Various assays can be used to test the functional capacity and the parameters of a candidate sortase molecule. For example, the ability of a candidate sortase molecule to mediate a transpeptidation reaction can be assessed by providing a moiety coupled to a sortase recognition motif, a fluorescently-labeled sortase acceptor motif, and the candidate sortase molecule in a reaction under conditions suitable for sortase activity. The generation of conjugates comprising the moiety and the fluorescent label, e.g., by gel separation and fluorescent imaging techniques, indicates the functional capacity of the candidate sortase molecule to mediate the transpeptidation reaction between a sortase recognition motif and a sortase acceptor motif. Other suitable assays for testing function and the parameters, e.g., calcium dependency and kinetics, are known in the art and are described herein, e.g., in Examples 1-4.

### Target Binding Molecule

Sortase based methods described herein can be used to attach a target binding molecule to another moiety, e.g., another polypeptide.

A target binding molecule refers to a molecule that has affinity for a target molecule. In an embodiment a target binding molecule can comprise, e.g., a binding partner, e.g., a ligand or receptor, from a ligand-receptor system. By way of example, a target binding molecule can be a soluble ligand or its receptor, e.g., a soluble extracellular domain of a receptor. In an embodiment, a target binding molecule comprises an antibody molecule, e.g., an antibody or antigen binding fragment thereof, single domain antibody (sdAb), or a single chain antibody (scFv). In an embodiment a target binding molecule comprises a non-antibody scaffold, e.g., a fibronectin, and the like. In embodiments, the target binding molecule is a single polypeptide. In embodiments, the target binding molecule comprises, one, two, or more, polypeptides. In embodiments, the target binding molecule is a polypeptide or fragment thereof of a naturally occurring protein expressed on a cell.

In embodiments, the target binding molecule comprises a non antibody scaffold, e.g., a fibronectin, ankyrin, domain antibody, lipocalin, small modular immuno-pharmaceutical, maxybody, Protein A, or affilin. The non antibody scaffold has the ability to bind to target, e.g., on a cell. In some embodiments, the target binding molecule comprises a non-antibody scaffold. A wide variety of non-antibody scaffolds can be employed so long as the resulting polypeptide includes at least one binding region which specifically binds to the target molecule on a target cell.

Non-antibody scaffolds include: fibronectin (Novartis, MA), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

Fibronectin scaffolds can be based on fibronectin type III domain (e.g., the tenth module of the fibronectin type III (¹⁰ Fn3 domain). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, this non-antibody scaffold mimics target binding properties that are similar in nature and affinity to those of antibodies. These scaffolds can be used in a loop randomization and shuffling strategy in vitro that is similar to the process of affinity maturation of antibodies in vivo.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as HER3. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Antibody Molecules

Sortase based methods described herein can be used to attach an antibody molecule to another moiety, e.g., another polypeptide.

An antibody molecule can be an immunoglobulin, e.g., an antibody, or an antigen binding portion thereof, e.g., a molecule that contain an antigen binding site which specifically binds an antigen, such as a polypeptide. Antibody molecules include “antibody fragments” which refers to a portion of an intact antibody that is sufficient to confer recognition and specific binding to a target antigen. Examples of antibody fragments include, but are not limited to, Fab, Fab′, F(ab′)2, and Fv fragments, linear antibodies, scFv antibodies, a linear antibody, single domain antibody (sdAb), e.g., either a variable light (VL) chain or a variable heavy (VH) chain, a camelid VHH domain, and multispecific antibodies formed from antibody fragments.

Antibody molecules can be polyclonal or monoclonal. The term “monoclonal” as applied to antibody molecules herein, refers to a population of antibody molecules that contain only one species of an antigen binding site capable of immunoreacting with a particular epitope.

In an embodiment, the antibody molecule is a “scFv,” which can comprise a fusion protein comprising a variable light (VL) chain and a variable heavy (VH) chain of an antibody, where the VH and VL are, e.g., linked via a short flexible polypeptide linker, e.g., a linker described herein. The scFv is capable of being expressed as a single chain polypeptide and retains the specificity of the intact antibody from which it is derived. Moreover, the VL and VH variable chains can be linked in either order, e.g., with respect to the N-terminal and C-terminal ends of the polypeptide, the scFv may comprise VL-linker-VH or may comprise VH-linker-VL. An scFv that can be prepared according to method known in the art (see, for example, Bird et al., (1988) Science 242:423-426 and Huston et al., (1988) Proc. Natl. Acad. Sci. USA 85:5879-5883).

As described above and elsewhere, scFv molecules can be produced by linking VH and VL chians together using flexible polypeptide linkers. In some embodiments, the scFv molecules comprise flexible polypeptide linker with an optimized length and/or amino acid composition. The flexible polypeptide linker length can greatly affect how the variable regions of a scFv fold and interact. In fact, if a short polypeptide linker is employed (e.g., between 5-10 amino acids), intrachain folding is prevented. For examples of linker orientation and size (see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT Publication Nos. WO2006/020258 and WO2007/024715, is incorporated herein by reference). In one embodiment, the peptide linker of the scFv consists of amino acids such as glycine and/or serine residues used alone or in combination, to link variable heavy and variable light chain regions together. In one embodiment, the flexible polypeptide linker is a Gly/Ser linker and, e.g., comprises the amino acid sequence (Gly-Gly-Gly-Ser)ₙ (SEQ ID NO: 43), where n is a positive integer equal to or greater than 1. For example, n=1, n=2, n=3. n=4, n=5 and n=6, n=7, n=8, n=9 and n=10. In one embodiment, the flexible polypeptide linkers include, but are not limited to, (Gly₄ Ser)₄ (SEQ ID NO: 44) or (Gly₄ Ser)₃ (SEQ ID NO: 45). In another embodiment, the linkers include multiple repeats of (Gly₂Ser), (GlySer) or (Gly₃Ser) (SEQ ID NO: 43).

In some embodiments, the antibody molecule is a single domain antibody (SDAB) molecules. Examples include, but are not limited to, heavy chain variable domains, binding molecules naturally devoid of light chains, single domains derived from conventional 4-chain antibodies, engineered domains and single domain scaffolds other than those derived from antibodies (e.g., described in more detail below). SDAB molecules may be any of the art, or any future single domain molecules. SDAB molecules may be derived from any species including, but not limited to mouse, human, camel, llama, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as a heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

In certain embodiments, the SDAB molecule is a single chain fusion polypeptide comprising one or more single domain molecules (e.g., nanobodies), devoid of a complementary variable domain or an immunoglobulin constant, e.g., Fc, region, that binds to one or more target antigens.

The SDAB molecules can be recombinant, CDR-grafted, humanized, camelized, de-immunized and/or in vitro generated (e.g., selected by phage display).

In one embodiment, the antibody molecule described herein comprises a human antibody or a fragment thereof.

In some embodiments, a non-human antibody is humanized, where specific sequences or regions of the antibody are modified to increase similarity to an antibody naturally produced in a human. In an embodiment, the antigen binding molecule is humanized.

### Methods for Sortase-Mediated Coupling

The methods presented herein relate to the coupling of a first moiety to a second moiety in a sortase-mediated reaction, using any of the sortase molecules described herein. In one embodiment, the first moiety is coupled to a sortase acceptor motif and the second moiety is coupled to a sortase recognition motif. Upon the addition of a sortase molecule described herein, the sortase cleaves a peptide bond in the sortase recognition motif, e.g., the peptide bond between a threonine and either a glycine or alanine, and forms an acyl-enzyme intermediate, e.g., a complex comprising the sortase molecule and the second moiety coupled to the cleaved sortase recognition motif. The acyl-enzyme intermediate reacts with the sortase acceptor motif coupled to the first moiety, e.g., by nucleophilic attack, and generates a peptide bond between the C-terminus of the sortase recognition motif and the N-terminus of the sortase acceptor motif. The resulting molecule comprises the second moiety coupled to the first moiety.

Reaction conditions for the cleavage and transfer of the second moiety coupled to the cleaved sortase recognition motif to the sortase acceptor motif coupled to the first moiety are similar to physiological conditions. The pH of the reaction can be between pH 4 and pH 10. Preferably, the pH is between pH 6 and pH 8. Most preferably, the pH is neutral, or around pH 7. The temperature of the reaction can be between 25° C. and 42° C. In some preferred embodiments, the temperature of the reaction is at or around body temperature, e.g., around 37° C. In some embodiments, the first moiety, the second moiety, and the sortase molecule are in solution in a reaction buffer. For example, the reaction buffer comprises buffering agents, e.g., sodium chloride, sodium bicarbonate, sodium phosphate, potassium chloride, magnesium chloride, and Tris. In one embodiment, the reaction buffer comprises a final concentration of 50 mM Tris-Cl, pH 7.4, and 150 mM NaCl. In other embodiments, the first moiety, the second moiety, and the sortase molecule are in cell culture media. Cell culture media may contain amino acids, vitamins (e.g., biotin, folic acid, niacinamide), D-glucose, reduced glutathione, various inorganic salts (e.g., calcium nitrate, potassium chloride, sodium chloride, sodium bicarbonate, etc), and fetal bovine serum. Optionally, the reaction buffer or cell culture media may contain calcium, e.g., between 0.1-10 mM calcium. In one embodiment, the reaction buffer does not contain any calcium. When the reaction is performed in cell culture, preferably no exogenous calcium is added to the cell culture reaction. The concentration of the sortase molecule and/or the second moiety can be added to the reaction in excess of the concentration of the first moiety for efficient catalysis.

The invention provides methods for labeling or generating fusion constructs at the surface of a cell. In one embodiment, the first moiety coupled to the sortase acceptor motif is disposed on the surface of a cell. The second moiety coupled to the sortase recognition motif and the sortase molecule (or the complex comprising the intermediate of the second moiety and the sortase molecule) is added to the cell culture media. After the sortase-mediated reaction, the coupled first moiety and second moiety are disposed on the surface of a cell. In some embodiments, the second moiety is a marker or a target binding molecule, and the sortase-mediated reaction functionalizes the cell for detection (i.e., by the signal generated from the marker), or targeted binding to a specific antigen.

In one embodiment, additional moieties coupled to sortase acceptor motifs and sortase recognition motifs wherein the structures and functions or the additional moieties are different, can be added to the reaction. This method allows the generation of multiple different fusion constructs in the same reaction, thereby facilitating e.g., a large plurality of combinations of moieties, e.g., a library of fusion proteins.

The present invention also provides methods utilizing more than one sortase, e.g., two sortase molecules, for coupling different moieties to generate at least two different coupled conjugates. Using two different sortases with different parameters, e.g., different sortase recognition motifs, or calcium dependence, allows control over the generation of specific combinations of moieties. In the case where the moieties coupled to the sortase acceptor motif are present on the surface of a cell, a cell can be produced with two different fusion proteins with different functions or markers.

For example, one sortase molecule can be utilized for the coupling of a first moiety to a second moiety, and another sortase molecule couples a third moiety to a fourth moiety. In one embodiment, the two sortase molecules are different, e.g., do not share significant sequence identity or homology. For example, one of the sortase molecules is a mutant sortase molecule described herein, while the other sortase molecule is a wild-type sortase molecule from a bacteria. Examples of wild-type sortases suitable for use in the methods described herein include, but are not limited to wild-type sortase molecules from Staphylococcus aureus, Streptococcus pyogenes, Actionomyces naeslundii, Bacillus anthracis, Bacillus cereus, Bacillus halodurans, Bacillus subtilis, Bifidobacterium longum, Clostridium botunlinum, Clostridium difficile, Corynebacterium diphtheriae, Corynebacterium efficiens, Corynebacterium glutamicum, Enterococcus faecium, Geobacillus sp. Listeria innocua, Listeria monocytogenes, Oceanobacillus iheyensis, Ruminococcus albus, Streptomyces avermitilis, Streptomyces coelicolor, Streptomyces griseus, Staphylococcus epidermis, Streptococcus agalactiae, Streptococcus equi, Streptococcus gordonii, Streptococcus pyogenes, Thermobifida fusca, or Tropheryma wipplei, or sortase molecule having at least 80, 85, 90, or 95% identity thereto. Further mutations may be introduced to the wild-type sortases described herein to further optimize reaction parameters, e.g., kinetics, calcium dependence, site specificity.

### Modifications and Homology

It will be understood by one of ordinary skill in the art that the sortase molecule of the invention may further be modified such that it varies in amino acid sequence, but not in desired activity. For example, additional nucleotide substitutions leading to amino acid substitutions at “non-essential” amino acid residues may be made to the protein For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made. Alternatively, the sortase molecule of the invention is further modified to vary in amino acid sequence and in desired activity, e.g., in the parameters described herein, e.g., reaction kinetics and calcium dependence.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Homology or identity, which are used interchangeably herein, refer to the level of similarity between two sequences, e.g., nucleic acid or amino acid sequences. To determine the percent homology or identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical or homologous at that position. The percent identity or homology between the two sequences is a function of the number of identical positions shared by the sequences (i.e., % identity=# of identical positions/total # of positions (e.g., overlapping positions)×100). In one embodiment the two sequences are the same length.

The determination of percent identity or homology between two sequences can be accomplished using a mathematical algorithm. Another, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity or homology between two sequences can be determined using techniques similar to those described above, with or without allowing gaps. In calculating percent identity or homology, only exact matches are counted.

In one aspect, the present invention contemplates modifications of the amino acid sequence of the sortase molecule described herein that generate functionally equivalent molecules. For example, the amino acid sequence of a sortase molecule described herein can be modified to retain at least about 60%, 61%, 62,%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity or homology of the starting amino acid sequence of the sortase molecule described herein. In an embodiment the sortase molecule has at least 60%, 61%, 62,%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity or homology with a sortase molecule described herein. In an embodiment the sortase molecule has at least 60% identity or homology with a sortase molecule described herein. In an embodiment, the sortase molecule has at least 70% identity or homology with a sortase molecule described herein. In an embodiment, the sortase molecule has at least 80% identity or homology with a sortase molecule described herein. In an embodiment, the sortase molecule has at least 85% identity or homology with a sortase molecule described herein. In an embodiment, the sortase molecule has at least 90% identity or homology with a sortase molecule described herein. In an embodiment, the sortase molecule has at least 95% identity or homology with a sortase molecule described herein. In an embodiment, the sortase molecule has at least 98% identity or homology with a sortase molecule described herein.

In an embodiment, the sortase molecule has at least 60%, 70%, 75%, 80%, 85%, 90%, 95% or 98% identity or homology with a sortase molecule described herein comprising a truncation of 59 amino acids at the N-terminus of SEQ ID NO: 3 and all seven of the following mutations: Pro94 mutated to Arg94 (abbreviated Pro94Arg or P94R), Glu105 mutated to Lys105 (abbreviated Glu105Lys or E105K), Glu108 mutated to Gln108 (abbreviated Glu108Gln or E108Q), Asp160 mutated to Asn160 (abbreviated Asp160Asn or D160N), Asp165 mutated to Ala165 (abbreviated Asp165Ala or D165A), Lys190 mutated to Glu190 (abbreviated Lys190Glu or K190E) and Lys196 mutated to Thr196 (abbreviated Lys196Thr or K196T), e.g., SEQ ID NO: 5.

### Nucleic Acid Molecules

Sortase Nucleic Acid Molecules

One aspect of the invention pertains to isolated nucleic acid molecules that encode a sortase molecule, including nucleic acids which encode a sortase molecule or a portion of such a polypeptide. As used herein, the term “nucleic acid molecule” includes DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

Nucleic acid molecules also include nucleic acid molecules sufficient for use as hybridization probes or primers to identify nucleic acid molecules that correspond to a sortase, e.g., those suitable for use as PCR primers for the amplification or mutation of nucleic acid molecules.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

A sortase nucleic acid molecule can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer. In another embodiment, a sortase nucleic acid molecule comprises a nucleic acid molecule which has a nucleotide sequence complementary to the nucleotide sequence of a sortase nucleic acid molecule or to the nucleotide sequence of a nucleic acid encoding a sortase protein. A nucleic acid molecule which is complementary to a given nucleotide sequence is one which is sufficiently complementary to the given nucleotide sequence that it can hybridize to the given nucleotide sequence thereby forming a stable duplex.

Moreover, a sortase nucleic acid molecule can comprise only a portion of a nucleic acid sequence, wherein the full length nucleic acid sequence encodes a sortase molecule. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, at least about 15, at least about 25, at least about 50, at least about 75, at least about 100, at least about 125, at least about 150, at least about 175, at least about 200, at least about 250, at least about 300, at least about 350, at least about 400, at least about 500, or at least about 600 or more consecutive nucleotides of a sortase nucleic acid molecule.

The invention further encompasses nucleic acid molecules that are substantially identical to the gene mutations and/or gene products described herein, such that they are at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or greater. In other embodiments, the invention further encompasses nucleic acid molecules that are substantially homologous to the sortase gene mutations and/or gene products described herein, such that they differ by only or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500, at least 600 nucleotides or any range in between.

The invention further encompasses nucleic acid molecules that are substantially identical to the gene mutations and/or gene products described herein, e.g., sortase nucleic acid molecule having a nucleotide sequence of SEQ ID NO:3, or encoding an amino acid sequence of SEQ ID NO:1) such that they are at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or greater. In other embodiments, the invention further encompasses nucleic acid molecules that are substantially homologous to the sortase nucleic acid molecule mutations and/or products thereof described herein, such that they differ by only or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100 nucleotides or any range in between.

In another embodiment, an isolated sortase nucleic acid molecule is at least 7, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 550, or more nucleotides in length and hybridizes under stringent conditions to a sortase nucleic acid molecule or to a nucleic acid molecule encoding a protein corresponding to a marker of the invention.

As used herein, the term “hybridizes under stringent conditions” is intended to describe conditions for hybridization and washing under which nucleotide sequences at least 60%, at least 65%, at least 70%, at least 75%, at least 80%, or at least 85% identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). Another, non-limiting example of stringent hybridization conditions are hybridization in 6× sodium chloride/sodium citrate (SSC) at about 45° C., followed by one or more washes in 0.2×SSC, 0.1% SDS at 50-65° C.

The invention also includes molecular beacon nucleic acid molecules having at least one region which is complementary to a sortase nucleic acid molecule, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

Other Nucleic Acid Molecules

Also encompassed by the invention are other nucleic acid molecules comprising a nucleic acid sequence encoding a sortase acceptor motif or a sortase recognition motif. In an embodiment, a nucleic acid molecule of the invention comprises a nucleic acid sequence encoding a moiety, e.g., a polypeptide, coupled to a sortase acceptor motif. In another embodiment, a nucleic acid molecule of the invention comprises a nucleic acid sequence encoding a moiety, e.g., a polypeptide, coupled to a sortase recognition motif.

### Expression Vectors, Host Cells and Recombinant Cells

In another aspect, the invention includes vectors (e.g., expression vectors), containing a nucleic acid encoding a sortase molecule described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. For cellular expression, one or more nucleic acids (e.g., cDNA or genomic DNA encoding a sortase molecule can be inserted into a replicable vector for cloning or for expression. Various vectors are publicly available. The vector can, for example, be a plasmid, cosmid, viral genome, phagemid, phage genome, or other autonomously replicating sequence. The appropriate coding nucleic acid sequence may be inserted into the vector by a variety of procedures known in the art. For example, appropriate restriction endonuclease sites can be engineered (e.g., using PCR). Then restriction digestion and ligation can be used to insert the coding nucleic acid sequence at an appropriate location.

A vector can include a sortase nucleic acid molecule in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors can be introduced into host cells to thereby produce a sortase molecule, including fusion proteins or polypeptides encoded by nucleic acids as described herein, mutant forms thereof, and the like). The expressed sortase molecules can be purified or isolated from the host cells and can be subsequently used in reactions in vitro or in cell culture to join a moiety, e.g., a polypeptide, to another moiety, polypeptide, or living cell, as described further herein.

The term “recombinant host cell” (or “host cell” or “recombinant cell”), as used herein, is intended to refer to a cell into which a recombinant expression vector, e.g., a sortase molecule expression vector, has been introduced. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein.

The recombinant expression vectors can be designed for expression of a sortase molecule in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. For bacterial expression, the sortase molecule can be produced with or without a signal sequence. For example, it can be produced within cells so that it accumulates in inclusion bodies, or in the soluble fraction. It can also be secreted, e.g., by addition of a prokaryotic signal sequence, e.g., an appropriate leader sequence such as from alkaline phosphatase, penicillinase, or heat-stable enterotoxin II.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria; the 2μ plasmid origin is suitable for yeast; and various viral origins (SV40, polyoma, adenovirus, VSV, or BPV) are useful for cloning vectors in mammalian cells.

Expression and cloning vectors typically contain a selection gene or marker. Typical selection genes encode proteins that (a) confer resistance to antibiotics or other toxins, e.g., ampicillin, neomycin, methotrexate, or tetracycline, (b) complement auxotrophic deficiencies (such as the URA3 marker in Saccharomyces), or (c) supply critical nutrients not available from complex media, e.g., the gene encoding D-alanine racemase for Bacilli. Various markers are also available for mammalian cells, e.g., DHFR or thymidine kinase. DHFR can be used in conjunction with a cell line (such as a CHO cell line) deficient in DHFR activity, prepared and propagated as described by Urlaub et al., Proc. Natl. Acad. Sci. USA, 77:4216 (1980).

Expression and cloning vectors usually contain a promoter operably linked to the nucleic acid sequence encoding the sortase molecule to direct mRNA synthesis. Exemplary promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems can also contain an appropriately located Shine-Dalgarno sequence. The T7 polymerase system can also be used to drive expression of a nucleic acid coding sequence placed under control of the T7 promoter. See, e.g., the pET vectors (EMD Chemicals, Gibbstown N.J., USA) and host cells, e.g., as described in Novagen User Protocol TB053 available from EMD Chemicals and U.S. Pat. No. 5,693,489. For example, such vectors can be used in combination with BL21(DE3) cells and BL21(DE3) pLysS cells to produce protein, e.g., at least 0.05, 0.1, or 0.3 mg per ml of cell culture. Other cells lines that can be used include DE3 lysogens of B834, BLR, HMS174, NovaBlue, including cells bearing a pLysS plasmid.

The sortase nucleic acid molecule can also be operably linked to a tag suitable for purification or isolation of the sortase molecule. Suitable tags for purification, isolation, or detection are known in the art, and include, but are not limited to, biotin, myc tag, histidine tags (e.g., 3×His, 6×His (SEQ ID NO: 32), 8×His (SEQ ID NO: 33)), hemagglutinin tag (HA tag), and fluorescent protein tags (e.g., GFP, RFP). For example, His tags comprise an amino acid motif of at least 3, at least 6, or at least 8 histidine residues and can be used for purification using nickel (Ni²) affinity columns. Use of such tags enables purification, e.g., through affinity purification or chromatography, of the expressed sortase molecule from the host cell for use in the methods further described herein.

In embodiments, the sortase molecule can be immobilized, for example, on a surface or support, for reactions that occur in solid phase.

The sortase molecule expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a sortase nucleic acid molecule within a recombinant expression vector or a sortase nucleic acid molecule containing sequences which allow it to homologous recombination into a specific site of the host cell's genome.

A host cell can be any prokaryotic or eukaryotic cell. For example, a sortase molecule can be expressed in bacterial cells (such as E. coli), insect cells, yeast or mammalian cells (such as Chinese hamster ovary cells (CHO) or COS cells, e.g., COS-7 cells, CV-1 origin SV40 cells; Gluzman (1981) Cell 23:175-182). Other suitable host cells are known to those skilled in the art. Exemplary bacterial host cells for expression include any transformable E. coli K-12 strain (such as E. coli BL21, C600, ATCC 23724; E. coli HB101 NRRLB-11371, ATCC-33694; E. coli MM294 ATCC-33625; E. coli W3110 ATCC-27325), strains of B. subtilis, Pseudomonas, and other bacilli.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (e.g., express) a sortase molecule. Accordingly, the invention further provides methods for producing a sortase molecule using the host cells. In one embodiment, the method includes culturing the host cell of the invention (into which a recombinant expression vector encoding a sortase molecule has been introduced) in a suitable medium such that a sortase molecule is produced. In another embodiment, the method further includes isolating a sortase molecule from the medium or the host cell.

In another aspect, the invention features, a cell or purified preparation of cells which include a sortase transgene, e.g., a nucleic acid molecule encoding the sortase molecules described herein. The cell preparation can consist of human or non-human cells, e.g., rodent cells, e.g., mouse or rat cells, rabbit cells, or pig cells. In embodiments, the cell or cells include a sortase transgene, e.g., a heterologous form of a sortase, e.g., a gene derived from humans (in the case of a non-human cell).

Also encompassed by the invention are other vectors comprising a nucleic acid sequence encoding a sortase acceptor motif or a sortase recognition motif. In an embodiment, a vector of the invention comprises a nucleic acid sequence encoding a moiety, e.g., a polypeptide, coupled to a sortase acceptor motif. In another embodiment, a vector of the invention comprises a nucleic acid sequence encoding a moiety, e.g., a polypeptide, coupled to a sortase recognition motif.

### Anti-Sortase Molecule Antibodies

Also disclosed herein is an antibody that is specific for a sortase mutant disclosed herein. An isolated sortase molecule, or a fragment thereof, can be used as an immunogen to generate antibodies using standard techniques for polyclonal and monoclonal antibody preparation. The full-length sortase molecule can be used or, alternatively, the invention provides antigenic peptide fragments for use as immunogens. The antigenic peptide of a sortase molecule comprises at least 8 (or at least 10, at least 15, at least 20, or at least 30 or more) amino acid residues of the amino acid sequence of one of the polypeptides of the invention, and encompasses an epitope of the protein such that an antibody raised against the peptide forms a specific immune complex with a marker of the invention to which the protein corresponds. Exemplary epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a sortase molecule described herein. In one embodiment, the antibody molecule specifically binds to a sortase molecule, e.g., specifically binds to an epitope formed by the sortase molecule.

An antibody directed against a sortase molecule (e.g., a monoclonal antibody) can be used to isolate the polypeptide by standard techniques, such as affinity chromatography or immunoprecipitation. Moreover, such an antibody can be used to detect the sortase molecule (e.g., in a cellular lysate or cell supernatant) in order to evaluate the level and pattern of expression of the sortase molecule. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

### Methods for Detection of Sortase Nucleic Acids and Molecules

Methods for evaluating nucleic acid encoding any of the sortase molecules described herein, mutations and/or gene products (e.g., the sortase molecule) thereof are known to those of skill in the art. In one embodiment, the nucleic acid encoding a sortase molecule is detected by a method chosen from one or more of: nucleic acid hybridization assay, amplification-based assays (e.g., polymerase chain reaction (PCR)), PCR-RFLP assay, real-time PCR, sequencing, screening analysis (including metaphase cytogenetic analysis by standard karyotype methods, FISH (e.g., break away FISH), spectral karyotyping or MFISH, comparative genomic hybridization), in situ hybridization, SSP, HPLC or mass-spectrometric genotyping.

Additional exemplary methods include, traditional “direct probe” methods such as Southern blots or in situ hybridization (e.g., fluorescence in situ hybridization (FISH) and FISH plus SKY), and “comparative probe” methods such as comparative genomic hybridization (CGH), e.g., cDNA-based or oligonucleotide-based CGH, can be used. The methods can be used in a wide variety of formats including, but not limited to, substrate (e.g., membrane or glass) bound methods or array-based approaches.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

In Vitro Characterization of the S. aureus Sortase a Mutant

The [P94R/E105K/E108Q/D160N/D165A/K190E/K196T] sortaseA mutant was expressed in E. coli and purified by affinity chromatography exploring the polyhistidine tag comprised at its C-terminus, following established protocols (Guimaraes et al., 2013). The introduced mutations did not seem to interfere with expression or protein folding as high yields of soluble, monodispersed protein were obtained (data not shown).

Characterization of the enzyme was initially done in vitro using purified proteins. As the reaction substrate, a scFV directed to CD19 (scFV19) comprising a sortase A recognition motif (LPETGG (SEQ ID NO: 46)) and a His8 (SEQ ID NO: 33) purification handle at the C-terminus (also referred to herein as scFv19.LPETGG.His8 (“LPETGG” and “His8” disclosed as SEQ ID NOS 46 and 33, respectively)) was cloned, expressed, and purified. This is the same scFV19 that was used in subsequent examples to test site-specific attachment to live cells using sortase:

(SEQ ID NO: 6)
METDTLLLWVLLLWVPGSTGEIVMTQSPATLSLSPGERATLSCRASQDISKYLNWYQQKPGQAPRLLIYHT
SRLHSGIPARFSGSGSGTDYTLTISSLQPEDFAVYFCQQGNTLPYTFGQGTKLEIKGGGGSGGGGSGGGGS
QVQLQESGPGLVKPSETLSLTCTVSGVSLPDYGVSWIRQPPGKGLEWIGVIWGSETTYYSSSLKSRVTISK

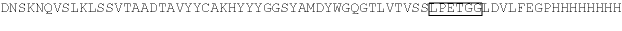

(The IgK signal peptide which is cleaved off co-translationally is underlined).

As a nucleophile for these test reactions fluorescently labeled peptide: GGGK(TAMRA) (KRUEGANA-001-EXP022) (SEQ ID NO:7) was synthesized and purified. The fluorophore moiety allowed for convenient monitoring of the reaction by SDS-PAGE followed by fluorescent scanning.

### Example 2

### The Mutant Sortase is Ca2+ Independent and Displays Fast Kinetics

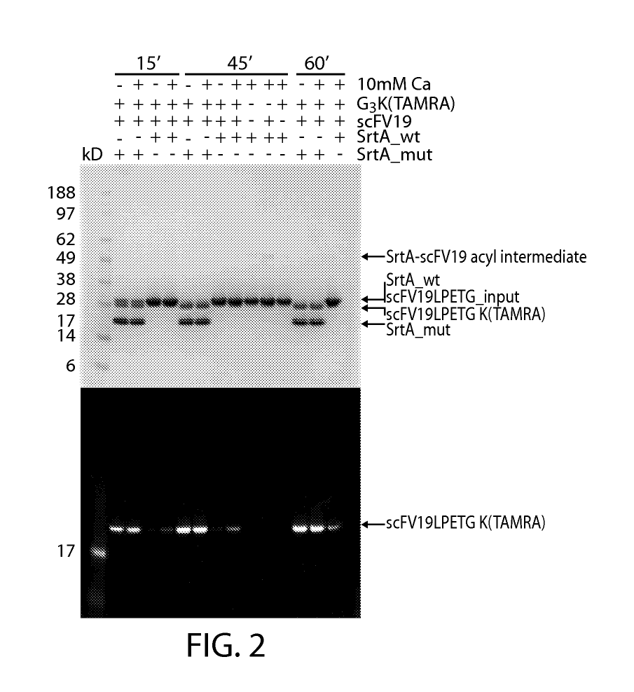

The activities of mutant and wild-type (SrtA aureus_His6SrtA26-206 (“His6” disclosed as SEQ ID NO: 32)) sortases were compared side-by-side in the absence or presence of 10 mM calcium in 50 mM Tris-Cl, pH 7.4, 150 mM NaCl buffer, using final concentrations of 40 μM sortase, 20 μM scFV.LPETG.His₈ (“LPETG” and “His₈” disclosed as SEQ ID NOS 39 and 33, respectively), and 1 mM GGGK(TAMRA) (SEQ ID NO:7). The reactions were incubated at 37° for different periods of time (as indicated in FIG. 2), and analyzed by reducing SDS-PAGE followed by fluorescent scanning (using a ChemiDoc gel imaging system from BioRad) and coomassie staining.

Only when sortase, scFV19, and the fluorescent peptide are incubated together, was fluorescent protein band detected, compatible with the size of the scFV19 conjugated to the TAMRA peptide (FIG. 2). This was true for the mutant sortase, regardless of whether calcium was present in the reaction mixture. Calcium was however essential for the activity of the wild-type sortase, as the labeled product was detected only if calcium was included in the buffer (FIG. 2). The mutant sortase was also faster. In both cases an increase in fluorescence was observed over time, but there was a clear distinction between the fluorescent intensities observed for the wild type and mutant enzymes. The mutant sortase demonstrated fluorescence as early as 15 minutes of incubation, while no fluorescence was detected at the same timepoint for the wild-type sortase reaction. Increased fluorescence was also detected for the reactions containing mutant sortase when compared to reactions containing wild-type sortase at all three timepoints. Under the reaction conditions described, labeling of the scFV19 with the TAMRA-decorated peptide and mutant sortase was complete after 45' incubation at 37° C.

### Example 3

### The Mutant Sortase a is Active in Cell Culture Media

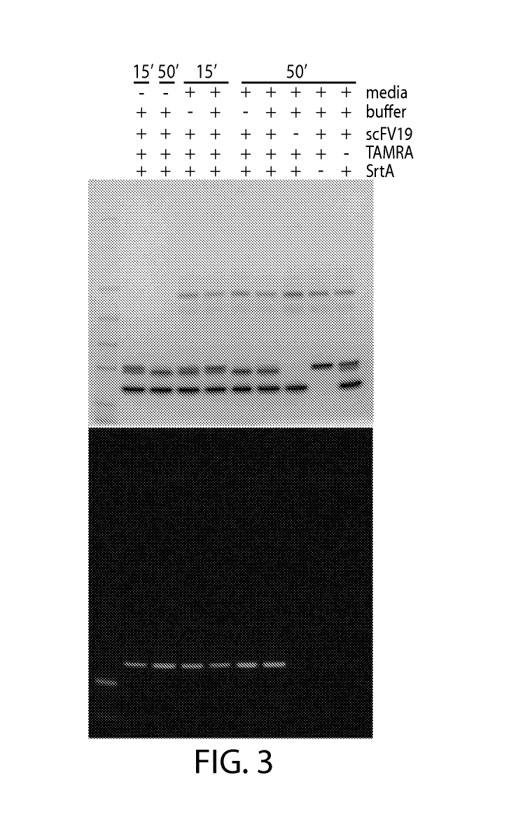

The activity of mutant sortase A was active in culture media (RMPI supplemented with 1% FBS) was determined using the same reaction conditions as in Example 2. The presence of the fluorescent bands indicate the successful coupling of scFv19 to the TAMRA-labeled peptide in the presence of cell culture media. No major labeling differences were detected between the reaction kinetics or the intensity of the fluorescence between reactions in buffer or in culture media. Thus, the results presented herein suggest the enzyme is also active in this culture media. As in Example 2, the reaction was complete upon 45′ incubation at 37° C. (FIG. 3). The results presented herein demonstrate the specificity of the reaction, as no proteins from the serum (detected upon coomassie staining) were labeled with a fluorophore.

### Example 4

### The Mutant Sortase a is Active in a Wide Range of Temperatures

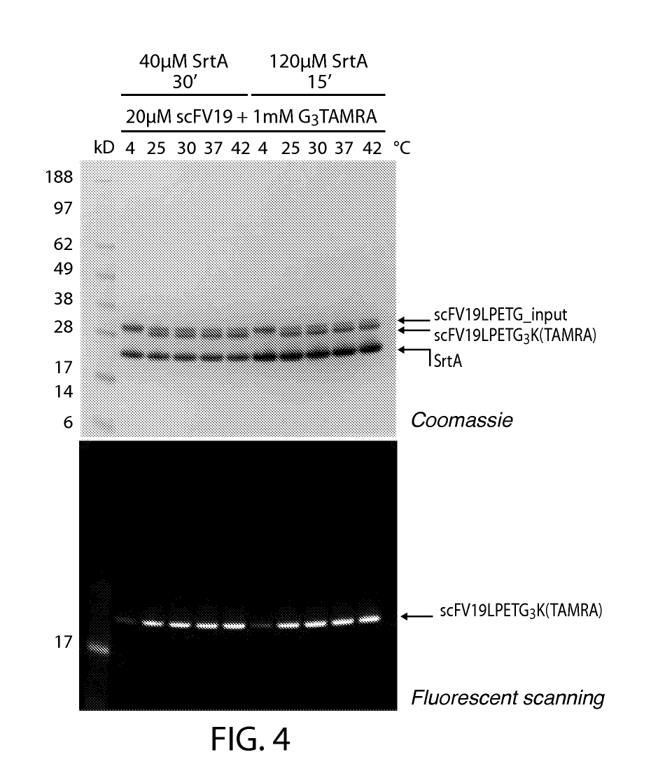

Because reaction temperature can influence enzyme activity, whether kinetics could be improved using temperatures above or below 37° C. was determined. The results presented herein demonstrate that the fluorescence was equivalent at each temperature point between 25 and 42° C., indicating that the mutant sortase A performed equally well at temperatures ranging from 25° C. to 42° C. (FIG. 4).

In this same experiment, whether the sortase concentration influences the reaction rate was also determined. The same labeling proportion in half of the time was observed, when using a three-fold higher concentration of enzyme (FIG. 4).

### Example 5

In Vitro Characterization of the scFV19 with a Sortase Receptor Motif

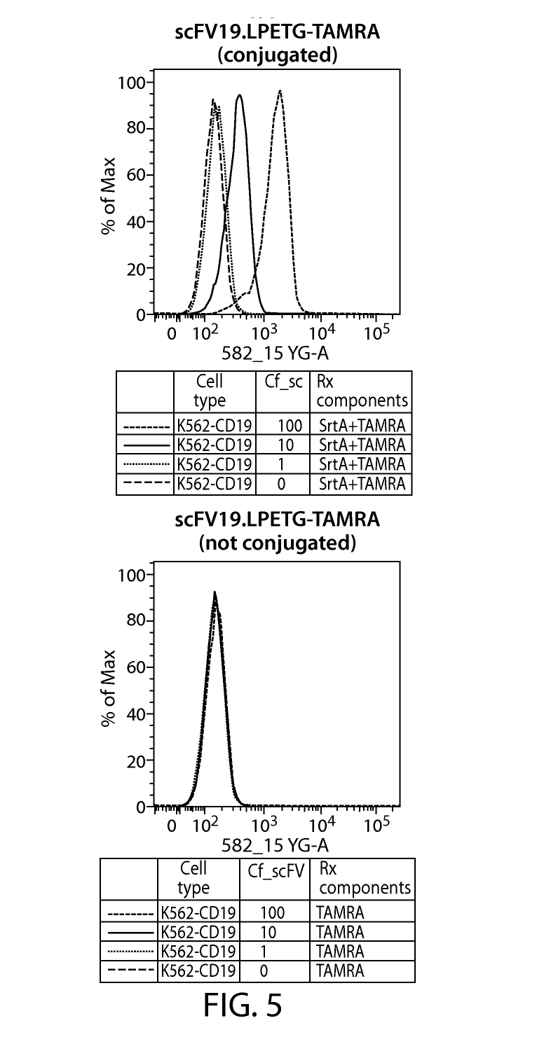

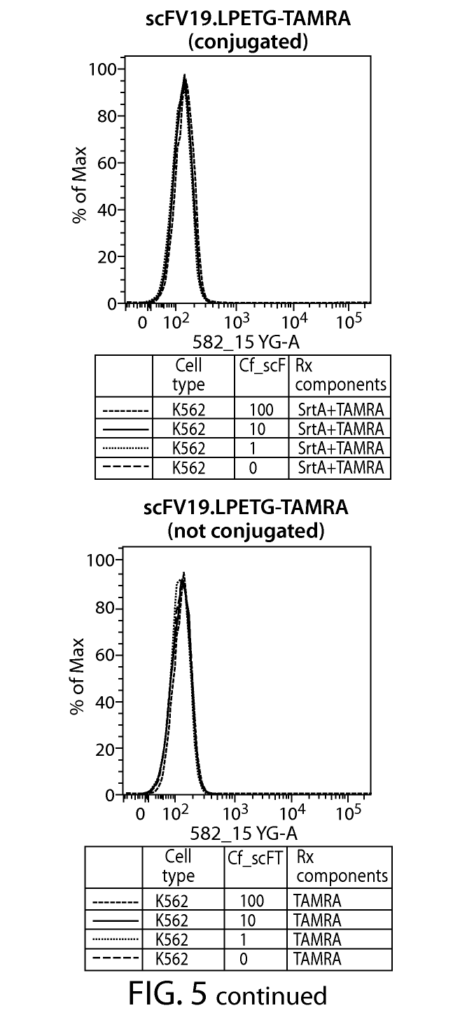

To determine whether the presence of the sortase-recognition motif interferes with the ability of the scFV19 to recognize CD19, the scFV19.LPETGG.His₈ (“LPETGG” and “His₈” disclosed as SEQ ID NOS 46 and 33, respectively) was labeled with the G₃K(TAMRA) peptide (SEQ ID NO:7) using the mutant sortase A as described in Example 1. A control reaction which did not include sortase was performed in parallel. Upon reaction, each of the preparations were filtered through a desalting column to remove unreacted G₃K(TAMRA) peptide (SEQ ID NO: 7). Different concentrations of the scFV19LPETG₃K(TAMRA) (“LPETG₃K” disclosed as SEQ ID NO: 49) conjugate and unconjugated control were then used to label untransduced K562 cells or K562 overexpressing CD19. It was shown by flow cytometry that cell labeling was observed only with the conjugate and only on K562 cells expressing CD19 (FIG. 5). These results demonstrated that the conjugation of the scFv19 molecule to the fluorescent TAMRA peptide by sortase did not interfere or impair scFv19 function, e.g., specific binding to CD19 expressed on the cell surface of K562 cells. Thus, the results presented herein confirm that the scFV19.LPETGG.His₈ substrate (“LPETGG” and “His₈” disclosed as SEQ ID NOS 46 and 33, respectively) for sortase is functional and that the sortase labeling strategy can be used to create new tools for FACS staining.

### Example 6

### Construction of an Fc-Apelin Conjugate Using Sortase:

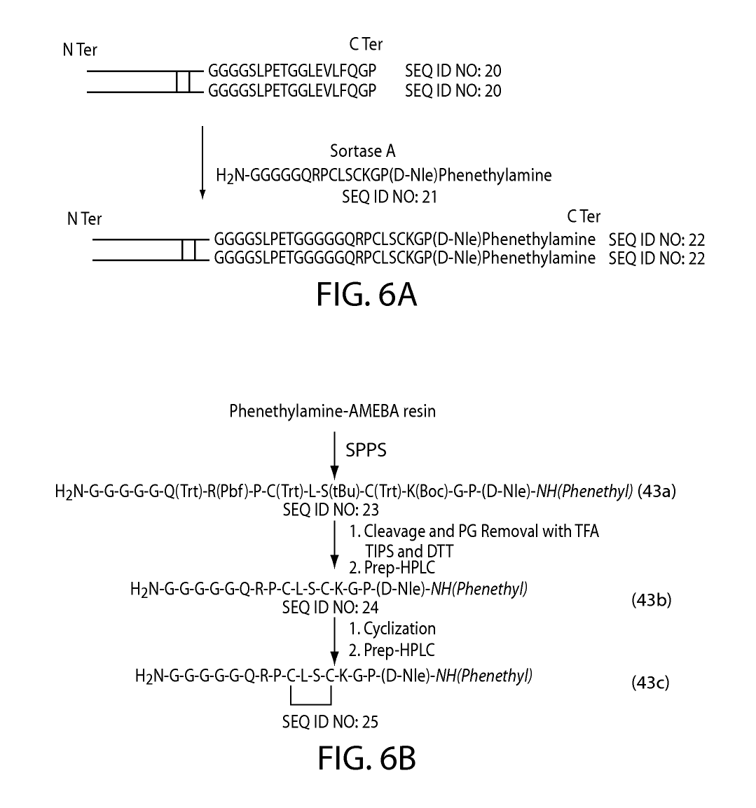

In this example, an Fc was conjugated to an apelin peptide using a sortase molecule described herein. The Fc peptide was generated with a sortase recognition motif at the C-terminus. The apelin peptide was generated with the sortase acceptor motif at the N-terminus. The [P94R/E105K/E108Q/D160N/D165A/K190E/K196T] mutant sortase A was incubated with the Fc peptide and the apelin peptide to produce an Fc-apelin conjugate. A schematic representation of this reaction is shown in FIG. 6A.

### Step 1: Preparation of Fc-Sortase-Recognition-Motif (Fc-SRM) Construct:

### Construct Cloning:

A DNA fragment containing the mouse Ig kappa chain signal peptide followed by a human Fc and a sortase recognition motif (LPXTG) (SEQ ID NO: 38) was codon optimized by gene synthesis (GeneArt) with 5′-NheI and 3′-EcoRI restriction sites. The resulting sequence was restriction digested with both NheI and EcoRI and ligated into NheI and EcoRI sites of vector pPL1146, downstream of a CMV promoter. The ligation was transformed into E coli DH5α cells and colonies containing the correct insert were identified by DNA sequencing. Sequence shown is for the sense strand and runs in the 5′ and 3′ direction.

The nucleic acid sequence of the Fc-sortase-recognition-motif molecule is as follows:

(SEQ ID NO: 8)
GCTAGCCACCATGGAAACCGACACCCTGCTGCTGTGGGTGCTGCTGCTGT
GGGTGCCAGGCAGCACCGGCGATAAGACCCACACCTGTCCTCCCTGTCCT
GCCCCTGAAGCTGCTGGCGGCCCTAGCGTGTTCCTGTTCCCCCCAAAGCC
CAAGGACACCCTGATGATCAGCCGGACCCCCGAAGTGACCTGCGTGGTGG
TGGATGTGTCCCACGAGGACCCTGAAGTGAAGTTCAATTGGTACGTGGAC
GGCGTGGAAGTGCACAACGCCAAGACCAAGCCCAGAGAGGAACAGTACAA
CAGCACCTACCGGGTGGTGTCCGTGCTGACCGTGCTGCACCAGGACTGGC
TGAACGGCAAAGAGTACAAGTGCAAGGTGTCCAACAAGGCCCTGCCAGCC
CCCATCGAGAAAACCATCAGCAAGGCCAAGGGCCAGCCCCGCGAACCCCA
GGTGTACACACTGCCCCCTAGCCGGGAAGAGATGACCAAGAACCAGGTGT
CCCTGACCTGTCTCGTGAAGGGCTTCTACCCCTCCGATATCGCCGTGGAA
TGGGAGAGCAACGGCCAGCCCGAGAACAACTACAAGACCACCCCCCCTGT
GCTGGACAGCGACGGCTCATTCTTCCTGTACAGCAAGCTGACAGTGGACA
AGAGCCGGTGGCAGCAGGGCAACGTGTTCAGCTGCAGCGTGATGCACGAG
GCCCTGCACAACCACTACACCCAGAAGTCCCTGAGCCTGAGCCCTGGAAA
AGGCGGCGGAGGCTCTCTGCCTGAAACAGGCGGACTGGAAGTGCTGTTCC
AGGGCCCCTAAGAATTC

The amino acid sequence of the Fc-sortase-recognition-motif molecule is as follows, wherein GGGGS (SEQ ID NO: 9) represents the linker and LPETGGLEVLFQGP (SEQ ID NO: 10) is the sortase recognition motif (and GGLEVLFQGP (SEQ ID NO: 11) is clipped during the sortase-mediated reaction):

| (SEQ ID NO: 12) | |
|---|---|
| 1 | METDTLLLWV LLLWVPGSTG DKTHTCPPCP APEAAGGPSV FLFPPKPKDT |
| 51 | LMISRTPEVT CVVVDVSHED PEVKFNWYVD GVEVHNAKTK PREEQYNSTY |
| 101 | RVVSVLTVLH QDWLNGKEYK CKVSNKALPA PIEKTISKAK GQPREPQVYT |
| 151 | LPPSREEMTK NQVSLTCLVK GFYPSDIAVE WESNGQPENN YKTTPPVLDS |
| 201 | DGSFFLYSKL TVDKSRWQQG NVFSCSVMHE ALHNHYTQKS LSLSPGKGGG |
| 251 | GSLPETGGLEVLFQGP |

In some embodiments, the linker has the sequence GGGS (SEQ ID NO: 43).

### Protein Expression and Purification:

Fc-SRM expression plasmid DNA was transfected into HEK293T cells at a density of 1×10⁶ cells per ml using standard polyethylenimine methods. 500 ml cultures were then grown in FreeStyle 293 Medium (Life Technologies) in 3 L flasks for 4 days at 37° C.

Fc-SRM protein was purified from clarified conditioned media. Briefly, 500 ml of conditioned media was flowed over a 5 ml HiTrap MabSelect SuRe column (GE Life Sciences) at 4 ml/min. The column was washed with 20 column volumes of PBS containing 0.1% Triton X-114 and then the Fc-sortase protein was eluted with 0.1M glycine, pH 2.7, neutralized with 1 M Tris-HCl, pH 9 and dialyzed against PBS. Protein yields were 10 to 20 mg per 500 ml conditioned media and endotoxin levels were <1 EU/mg as measured by the Charles River ENDOSAFE PTS test.

The following assays were performed for quality control of the Fc-SRM protein:

LC/MS of native Fc-SRM protein: Peak was heterogeneous and about 3 kDa larger than expected for dimers. This is characteristic of N-linked glycosylation expected for Fc which has a consensus N-linked glycosylation site.
LC/MS of reduced, N-deglycosylated Fc-SRM protein: Peak was sharp. The molecular weight was 2 daltons less than theoretical, likely due to Cysteine ×2 reduction.
Analytical size exclusion on Superdex 200: Fc-SRM protein had between 89 and 100% dimer, 0 to 10% tetramer, and 0 to 1% aggregate.
Reducing SDS/PAGE: The protein migrated predominately as a monomer of the expected size.

### Step 2: Preparation of Apelin Peptide (H₂N-GGGGGQRPC*LSC*KGP(D-Nle)Phenethylamine)(SEQ ID NO: 13) for Sortase Conjugation

A schematic representation of this step is shown in FIG. 6B.

### Step 2a: Preparation of Intermediate 43a

Phenethylamine-AMEBA resin (Sigma Aldrich, 0.25 g, 0.25 mmol, 1.0 mmol/g) was subjected to solid phase peptide synthesis on an automatic peptide synthesizer (CEM LIBERTY) with standard double Arg for the Arg residues. Amino acids were prepared as 0.2 M solutions in DMF.

A coupling cycle was defined as follows:
Amino acid coupling: AA (4.0 eq.), HATU (4.0 eq.), DIEA (25 eq.)
Washing: DMF (3×10 mL, 1 min each time).
Fmoc deprotection: Piperidine/DMF (1:4) (10 mL, 75° C. for 1 min, then 10 mL, 75° C. for 3 min).
Washing: DMF (4×10 mL, 1 min each time).

| | | Number of | |
|---|---|---|---|
| | | couplings × | Reaction |
| Coupling | AA | Reaction time | Temperature |
| 1 | Fmoc-D-Nle-OH | 1 × 5 min | 75° C. |
| 2 | Fmoc-L-Pro-OH | 1 × 5 min | 75° C. |
| 3 | Fmoc-Gly-OH | 1 × 5 min | 75° C. |
| 4 | Fmoc-L-Lys(Boc)-OH | 1 × 5 min | 75° C. |
| 5 | Fmoc-L-Cys(Trt)-OH | 1 × 6 min | 2 min at 25° C. |
| | | | 4 min at 50° C. |
| 6 | Fmoc-L-Ser(tBu)-OH | 1 × 5 min | 75° C. |
| 7 | Fmoc-L-Leu-OH | 1 × 5 min | 75° C. |
| 8 | Fmoc-L-Cys(Trt)-OH | 1 × 6 min | 2 min at 25° C. |
| | | | 4 min at 50° C. |
| 9 | Fmoc-L-Pro-OH | 1 × 5 min | 75 |
| 10 | Fmoc-L-Arg(Pbf)-OH | 2 × 30 min | 25 min at 25° C. |
| | | | 5 min at 75° C. |
| 11 | Fmoc-L-Gln(Trt)-OH | 1 × 5 min | 75° C. |
| 12 | Fmoc-Gly-Gly-Gly- | 1 × 5 min | 75° C. |
| | OH | | |
| 13 | Fmoc-Gly-OH | 1 × 5 min | 75° C. |
| 14 | Fmoc-Gly-OH | 1 × 5 min | 75° C. |

After the assembly of the peptide, the resin was washed with DMF (3×10 mL), DCM (3×10 mL). The peptide resin was dried under vacuum at room temperature to give Intermediate 43a (0.622 g, 0.25 mmol).

### Step 2b: Preparation of Intermediate 42b, H₂N-G-G-G-G-G-Q-R-P-C-L-S-C-K-G-P-(D-Nle)-NH(Phenethyl) (SEQ ID NO: 13)

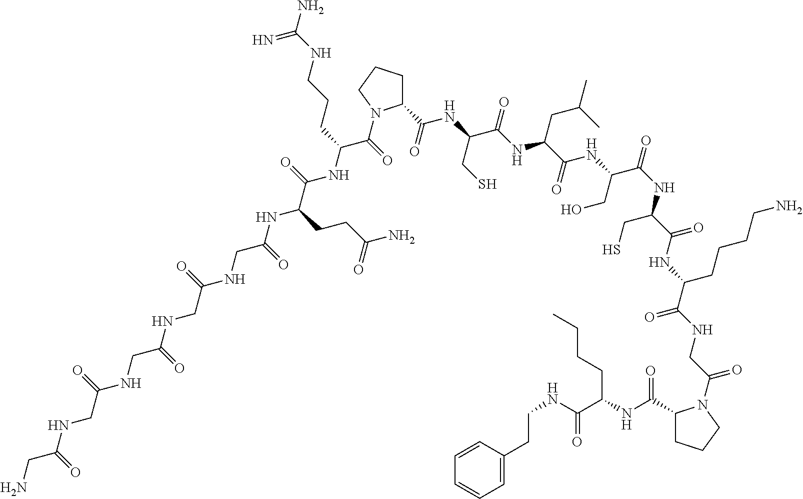

### 1) Cleavage and Protecting Group Removal

To intermediate 43a (0.622 g, 0.25 mmol) was added 3 mL solution of 95% TFA/2.5% H₂O/2.5% TIPS and DTT (771 mg, 5.00 mmol), the resulting mixture was shaken at room temperature for 3 hours, then filtered. The filtrate was dropped into 40 mL of cold ether, then centrifuged at 4000 rpm for 5 minutes. The solvent was removed and the white solid was washed with ether (3×40 mL), vortexed and centrifuged. The solid was dried under high vacuum at 25° C. for 1 hour.

### 2) Purification

The above white solid was then purified by preparative HPLC (Sunfire™ Prep C18 OBD™ 30×50 mm Sum column ACN/H₂O w/0.1% TFA 75 ml/min, 10-30% ACN 8 min gradient). The product fraction was lyophilized to give intermediate 43b as TFA salt (44 mg, 11%).

Step 2c: Preparation of H₂N-G-G-G-G-G-Q-R-P-C*-L-S-C*-K-G-P-(D-Nle)-NH(Phenethyl) (Disulfide C⁹-C¹²) (SEQ ID NO: 13), Intermediate 43c

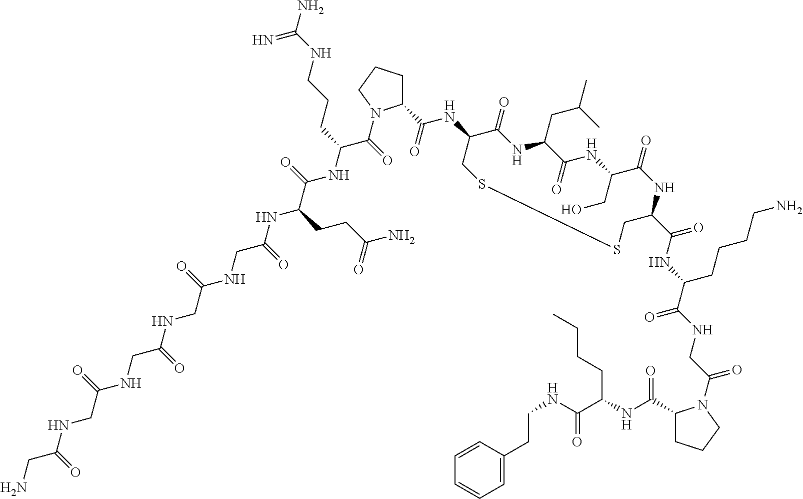

To intermediate 43b (44 mg, 0.028 mmol) in 0.9 mL of H₂O was added I₂ (50 mM in AcOH, 1.1 mL 0.055 mmol) dropwise. The mixture was shaken at room temperature overnight. LC/MS showed the reaction completed. To the reaction mixture was added several drops of 0.5 M of ascorbic acid solution (MeOH/H₂O=1/1) until the color of the solution disappeared. The mixture was diluted with MeOH for HPLC purification. The purification was carried out by preparative HPLC (Sunfire™ Prep C18 OBD® 30×50 mm Sum column ACN/H2O w/0.1% TFA 75 ml/min, 10-30% ACN 8 min gradient). The product fraction was lyophilized to give H₂N-G-G-G-G-G-Q-R-P-C*-L-S-C*-K-G-P-(D-Nle)-NH(Phenethyl) (disulfide C⁹-C¹²) (SEQ ID NO: 13), intermediate 43c as TFA salt (13 mg, 30%). LC/MS (QT2, ProductAnalysis-HRMS-Acidic, Waters Acquity UPLC BEH C18 1.7 um 2.1×50 mm, 50° C., Eluent A: Water+0.1% Formic Acid, Eluent B: Acetonitrile+0.1% Formic Acid, gradient 2% to 98% B/A over 5.15 mins): Retention time: 0.98 mins; MS [M+2]^{2±}: observed: 1587.7993, calculated: 1587.868.

### Step 3: Sortase Conjugation of Fc-Sortase-Recognition-Motif and Intermediate 43c

### 1) Chemoenzymatic Sortase Conjugation

On ice bath, to the Fc-SRM (698 μl, 0.040 μmol, 3.15 mg/mL) in PBS (pH7.4) buffer solution was added the solution of H₂N-G-G-G-G-G-Q-R-P-C*-L-S-C*-K-G-P-(D-Nle)-NH(Phenethyl) (disulfide C⁹-C¹²) (SEQ ID NO: 13) (64.1 μL, 2.018 μmoL, 50 mg/mL) (SEQ ID NO: 13) in Tris-8.0 buffer, followed by 520 μM of sortase A (78 μL, 0.040 μmoL in 50 mM Tris-Cl pH7.4, 150 mM NaCl. The mixture was shaken at room temperature overnight. LC/MS showed the reaction completed and that Fc-apelin conjugate was successfully generated.

### 2) Purification and Desalting

The above solution was flowed over a 5 mL HiTrap Mab Select SuRe column (GE Lifesciences #11-0034-95) at 4 mL/min on ATTA XPRESS. The conjugate protein was washed on the column with 20 column volumes (CV) PBS+0.1% Triton 114 and eluted with 0.1M glycine, pH 2.7, neutralized with 1 M tris-HCl, pH 9 and dialyzed versus PBS. The purified solution was desalted by using Zeba Spin Desalting Column, 5 mL (89891) to give 1.5 mL target solution, the average concentration was 0.598 mg/mL, and the recoverage was 90%. LCMS (QT2, Protein_20-70 kDa_3 min, AcQuity ProSwift RP-3U 4.6×50 mm, 1.0 mL/min, Eluent A: Water+0.1% Formic Acid, Eluent B: Acetonitrile+0.1% Formic Acid, gradient 2% to 98% B/A over 3 mins): Rₜ=1.55 minutes, MS [M+H] 58845.0000.

The amino acid sequence of the Fc-apelin conjugate is provided below:

| (SEQ ID NO: 14) | |
|---|---|
| 1 | METDTLLLWV LLLWVPGSTG DKTHTCPPCP APEAAGGPSV FLFPPKPKDT |
| 51 | LMISRTPEVT CVVVDVSHED PEVKFNWYVD GVEVHNAKTK PREEQYNSTY |
| 101 | RVVSVLTVLH QDWLNGKEYK CKVSNKALPA PIEKTISKAK GQPREPQVYT |
| 151 | LPPSREEMTK NQVSLTCLVK GFYPSDIAVE WESNGQPENN YKTTPPVLDS |
| 201 | DGSFFLYSKL TVDKSRWQQG NVFSCSVMHE ALHNHYTQKS LSLSPGKGGG |
| 251 | GSLPETGGGGGQRPC*LSC*KGP (D-Nle) Phenethylamine |

wherein LSLSPGKGGG GSLPETGGGGG (SEQ ID NO: 47) represents the linker and QRPC*LSC*KGP(D-Nle)Phenethylamine (SEQ ID NO: 48) represents the apelin polypeptide.

Other sortase mutants, as described herein, can also be used with the same reaction conditions as described in this example to generate a conjugate molecule, e.g., an Fc-apelin conjugate.

### Example 7

### Construction of a Second Fc-Apelin Conjugate Using Sortase.

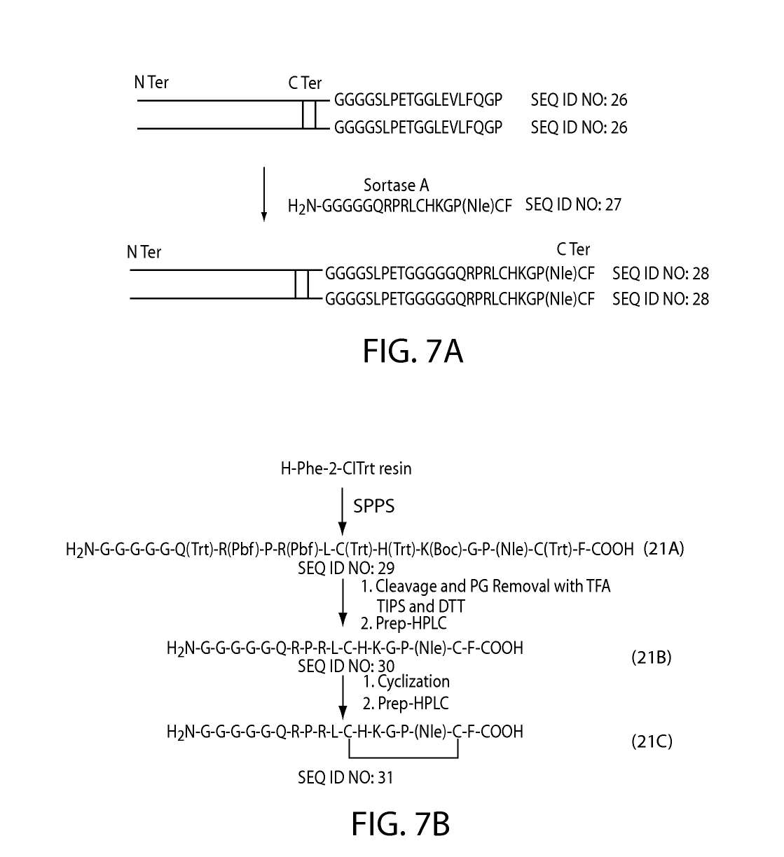

In this example, an Fc peptide was conjugated to a second apelin peptide using a sortase molecule as described herein. The Fc peptide was generated with a sortase recognition motif at the C-terminus. The apelin peptide was generated with a sortase acceptor motif at the N-terminus. A [P94R/E105K/E108Q/D160N/D165A/K190E/K196T] mutant sortase A was incubated with the Fc peptide and the apelin peptide to produce an Fc-apelin conjugate. A schematic representation of this reaction is shown in FIG. 7A. The reaction conditions were similar to those described in Example 6, however the apelin peptide used in this example is different from the peptide utilized in Example 6.

### Step 1: Preparation of Fc-Sortase-Recognition-Motif (Fc-SRM) Construct:

### Construct Cloning:

A DNA fragment containing the mouse Ig kappa chain signal peptide followed by a human Fc and a sortase recognition motif (LPXTG) (SEQ ID NO: 38) was codon optimized by gene synthesis (GeneArt) with 5′-NheI and 3′-EcoRI restriction sites. The resulting sequence was restriction digested with both NheI and EcoRI and ligated into NheI and EcoRI sites of vector pPL1146, downstream of a CMV promoter. The ligation was transformed into E coli DH5α cells and colonies containing the correct insert were identified by DNA sequencing. Sequence shown is for the sense strand and runs in the 5′ and 3′ direction.

The nucleic acid sequence of the Fc-SRM is as follows:

(SEQ ID NO: 8)
GCTAGCCACCATGGAAACCGACACCCTGCTGCTGTGGGTGCTGCTGCTGT
GGGTGCCAGGCAGCACCGGCGATAAGACCCACACCTGTCCTCCCTGTCCT
GCCCCTGAAGCTGCTGGCGGCCCTAGCGTGTTCCTGTTCCCCCCAAAGCC
CAAGGACACCCTGATGATCAGCCGGACCCCCGAAGTGACCTGCGTGGTGG
TGGATGTGTCCCACGAGGACCCTGAAGTGAAGTTCAATTGGTACGTGGAC
GGCGTGGAAGTGCACAACGCCAAGACCAAGCCCAGAGAGGAACAGTACAA
CAGCACCTACCGGGTGGTGTCCGTGCTGACCGTGCTGCACCAGGACTGGC
TGAACGGCAAAGAGTACAAGTGCAAGGTGTCCAACAAGGCCCTGCCAGCC
CCCATCGAGAAAACCATCAGCAAGGCCAAGGGCCAGCCCCGCGAACCCCA
GGTGTACACACTGCCCCCTAGCCGGGAAGAGATGACCAAGAACCAGGTGT
CCCTGACCTGTCTCGTGAAGGGCTTCTACCCCTCCGATATCGCCGTGGAA
TGGGAGAGCAACGGCCAGCCCGAGAACAACTACAAGACCACCCCCCCTGT
GCTGGACAGCGACGGCTCATTCTTCCTGTACAGCAAGCTGACAGTGGACA
AGAGCCGGTGGCAGCAGGGCAACGTGTTCAGCTGCAGCGTGATGCACGAG
GCCCTGCACAACCACTACACCCAGAAGTCCCTGAGCCTGAGCCCTGGAAA
AGGCGGCGGAGGCTCTCTGCCTGAAACAGGCGGACTGGAAGTGCTGTTCC
AGGGCCCCTAAGAATTC

The amino acid sequence of the Fc-SRM is as follows:

| (SEQ ID NO: 12) | |
|---|---|
| 1 | METDTLLLWV LLLWVPGSTG DKTHTCPPCP APEAAGGPSV FLFPPKPKDT |
| 51 | LMISRTPEVT CVVVDVSHED PEVKFNWYVD GVEVHNAKTK PREEQYNSTY |
| 101 | RVVSVLTVLH QDWLNGKEYK CKVSNKALPA PIEKTISKAK GQPREPQVYT |
| 151 | LPPSREEMTK NQVSLTCLVK GFYPSDIAVE WESNGQPENN YKTTPPVLDS |
| 201 | DGSFFLYSKL TVDKSRWQQG NVFSCSVMHE ALHNHYTQKS LSLSPGKGGG |
| 251 | GSLPETGGLEVLFQGP |

wherein GGGGS (SEQ ID NO: 9) represents the linker and LPETGGLEVLFQGP (SEQ ID NO: 10) the sortase recognition motif (note: the GGLEVLFQGP(SEQ ID NO: 11) \ is clipped during sortase treatment).

### Protein Expression and Purification:

Fc-SRM expression plasmid DNA was transfected into HEK293T cells at a density of 1×10⁶ cells per ml using standard polyethylenimine methods. 500 ml cultures were then grown in FreeStyle 293 Medium (Life Technologies) in 3 L flasks for 4 days at 37° C.

Fc-SRM protein was purified from clarified conditioned media. Briefly, 500 ml of conditioned media was flowed over a 5 ml HiTrap MabSelect SuRe column (GE Life Sciences) at 4 ml/min. The column was washed with 20 column volumes of PBS containing 0.1% Triton X-114 and then the Fc-sortase protein was eluted with 0.1M glycine, pH 2.7, neutralized with 1 M Tris-HCl, pH 9 and dialyzed against PBS. Protein yields were 10 to 20 mg per 500 ml conditioned media and endotoxin levels were <1 EU/mg as measured by the Charles River ENDOSAFE PTS test.

The following assays were performed for quality control of the Fc-SRM protein:

LC/MS of native Fc-SRM protein: Peak was heterogeneous and about 3 kDa larger than expected for dimers. This is characteristic of N-linked glycosylation expected for Fc which has a consensus N-linked glycosylation site.
LC/MS of reduced, N-deglycosylated Fc-SRM protein: Peak was sharp. The molecular weight was 2 daltons less than theoretical, likely due to Cysteine ×2 reduction.
Analytical size exclusion on Superdex 200: Fc-SRM protein had between 89 and 100% dimer, 0 to 10% tetramer, and 0 to 1% aggregate.
Reducing SDS/PAGE: The protein migrated predominately as a monomer of the expected size.
Step 2: Preparation of Apelin peptide H₂N-GGGGGQRPRLC*HKGP(Nle)C*F—COOH (SEQ ID NO: 15) for Sortase conjugation

A schematic representation of this step is shown in FIG. 7B.

### Step 2a: Preparation of Intermediate 21A

Two batches of H-Phe-2-C1Trt resin (Novabiochem, 0.342 g, 0.25 mmol, 0.73 mmol/g) were subjected to solid phase peptide synthesis on an automatic peptide synthesizer (CEM LIBERTY) with standard double Arg for the Arg residues. Amino acids were prepared as 0.2 M solutions in DMF.

A coupling cycle was defined as follows:
Amino acid coupling: AA (4.0 eq.), HATU (4.0 eq.), DIEA (25 eq.)
Washing: DMF (3×10 mL, 1 min each time).
Fmoc deprotection: Piperidine/DMF (1:4) (10 mL, 75° C. for 1 min, then 10 mL, 75° C. for 3 min).
Washing: DMF (4×10 mL, 1 min each time).

| | | Number of | |
|---|---|---|---|
| | | couplings × | Reaction |
| Coupling | AA | Reaction time | Temperature |
| 1 | Fmoc-L-Cys(Trt)-OH | 1 × 6 min | 2 min at 25° C. |
| | | | 4 min at 50° C. |
| 2 | Fmoc-L-Nle-OH | 1 × 5 min | 75° C. |
| 3 | Fmoc-L-Pro-OH | 1 × 5 min | 75° C. |
| 4 | Fmoc-L-Gly-OH | 1 × 5 min | 75° C. |
| 5 | Fmoc-Lys(Boc)-OH | 1 × 5 min | 75° C. |
| 6 | Fmoc-L-His(Trt)-OH | 1 × 5 min | 75° C. |
| 7 | Fmoc-L-Cys(Trt)-OH | 1 × 6 min | 2 min at 25° C. |
| | | | 4 min at 50° C. |
| 8 | Fmoc-L-Leu-OH | 1 × 5 min | 75° C. |
| 9 | Fmoc-L-Arg(Pbf)-OH | 2 × 30 min | 25 min at 25° C. |
| | | | 5 min at 75° C. |
| 10 | Fmoc-L-Pro-OH | 1 × 5 min | 75° C. |
| 11 | Fmoc-L-Arg(Pbf)-OH | 2 × 30 min | 25 min at 25° C. |
| | | | 5 min at 75° C. |
| 12 | Fmoc-L-Gln(Trt)-OH | 1 × 5 min | 75° C. |
| 13 | Fmoc-Gly-Gly-Gly- | 1 × 5 min | 75° C. |
| | OH | | |
| 14 | Fmoc-Gly-OH | 1 × 5 min | 75° C. |
| 15 | Fmoc-Gly-OH | 1 × 5 min | 75° C. |

After the assembly of the peptide, each batch of resin was washed with DMF (3×10 mL), DCM (3×10 mL). The combined peptide resin was dried under vacuum at room temperature to give Intermediate 21A, (1.454 g, 0.5 mmol).

### Step 2b: Preparation of Intermediate 21B, H₂N-GGGGGQRPRLCHKGP(Nle)CF—COOH (SEQ ID NO: 15)

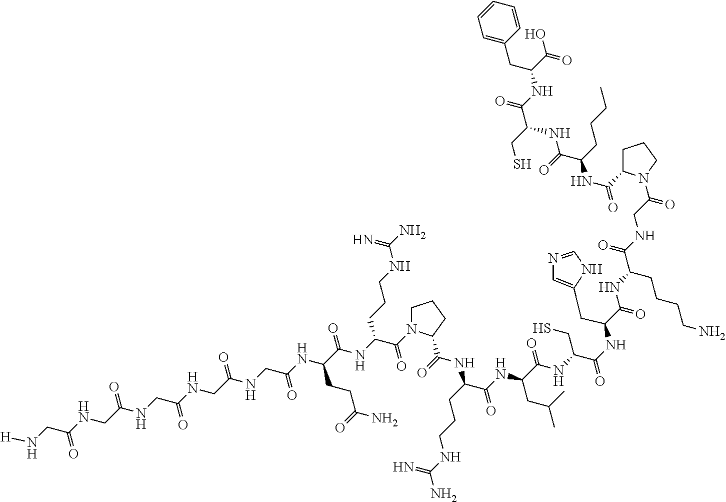 [SMILES: CCCC[C@@H](NC(=O)[C@@H]1CCCN1C(=O)CNC(=O)[C@H](CCCCN)NC(=O)[C@H](Cc1cnc[nH]1)NC(=O)[C@@H](CS)NC(=O)[C@@H](CC(C)C)NC(=O)[C@@H](CCCNC(=N)N)NC(=O)[C@H]1CCCN1C(=O)[C@@H](CCCNC(=N)N)NC(=O)[C@@H](CCC(N)=O)NC(=O)CNC(=O)CNC(=O)CNC(=O)CNC(=O)CN)C(=O)N[C@H](CS)C(=O)N[C@H](Cc1ccccc1)C(=O)O]

### 1) Cleavage and Protecting Group Removal

To intermediate 21A (1.454 g, 0.5 mmol) was added 6 mL solution of 95% TFA/2.5% H₂O/2.5% TIPS and DTT (1.452 g, 10.00 mmol), the resulting mixture was shaken at room temperature for 3 hours, then filtered. The filtrate was dropped into 80 mL of cold ether, then centrifuged at 4000 rpm for 5 minutes. The solvent was removed and the white solid was washed with ether (3×80 mL), vortexed and centrifuged. The solid was dried under high vacuum at 25° C. for 1 hour.

### 2) Purification

The above white solid was then purified by preparative HPLC (Sunfire™ Prep C18 OBD™ 30×50 mm 5 um column ACN/H₂O w/0.1% TFA 75 ml/min, 10-30% ACN 8 min gradient). The product fraction was lyophilized to give intermediate 21B as TFA salt (213 mg, 23%).

Step 2c: Preparation of H₂N-GGGGGQRPRLC*HKGP(Nle)C*F—COOH (Disulfide C¹¹-C¹⁷) (SEQ ID NO: 15), Intermediate 21C

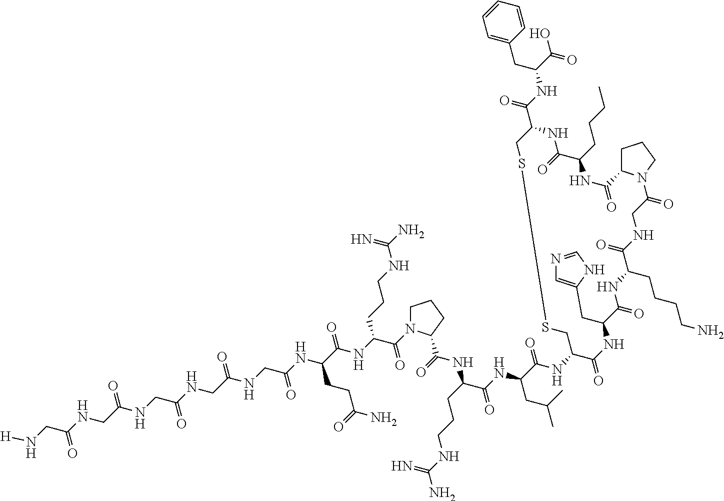 [SMILES: CCCC[C@H]1NC(=O)[C@@H]2CCCN2C(=O)CNC(=O)[C@H](CCCCN)NC(=O)[C@H](Cc2cnc[nH]2)NC(=O)[C@H](NC(=O)[C@@H](CC(C)C)NC(=O)[C@@H](CCCNC(=N)N)NC(=O)[C@H]2CCCN2C(=O)[C@@H](CCCNC(=N)N)NC(=O)[C@@H](CCC(N)=O)NC(=O)CNC(=O)CNC(=O)CNC(=O)CNC(=O)CN)CSSC[C@H](C(=O)N[C@H](Cc2ccccc2)C(=O)O)NC1=O]

To intermediate 21B (213 mg, 0.166 mmol) in 3.85 mL of H₂O was added I₂ (50 mM in AcOH, 4.63 mL, 0.232 mmol) dropwise. The mixture was shaken at room temperature overnight. LC/MS showed the reaction completed. To the reaction mixture was added several drops of 0.5 M of ascorbic acid solution (MeOH/H₂O=1/1) until the color of the solution disappeared. The mixture was diluted with MeOH for HPLC purification. The purification was carried out by preparative HPLC (Sunfire™ Prep C18 OBD™ 30×50 mm 5 um column ACN/H2O w/0.1% TFA 75 ml/min, 7.5-20% ACN 8 min gradient). The product fraction was lyophilized to give H₂N-GGGGGQRPRLC*HKGP(Nle)C*F—COOH (disulfide C¹¹-C¹⁷) (SEQ ID NO: 15), intermediate 21C as TFA salt (65 mg, 31%). LC/MS (QT2, ProductAnalysis-HRMS-Acidic, Waters Acquity UPLC BEH C18 1.7 um 2.1×50 mm, 50° C., Eluent A: Water+0.1% Formic Acid, Eluent B: Acetonitrile+0.1% Formic Acid, gradient 2% to 98% B/A over 5.15 mins): Retention time: 0.79 mins; MS [M+2]^{2±}: observed: 919.9562.

### Step 3: Sortase Conjugation of Fc-Sortase and Intermediate 21C

### 1) Chemoenzymatic Sortase Conjugation

On ice bath, to the FC-SRM (1397 μl, 0.081 μmol) in PBS (pH7.4) buffer solution was added the solution of H₂N-GGGGGQRPRLC*HKGP(Nle)C*F—COOH (disulfide C¹¹-C¹⁷) (SEQ ID NO: 15) (148 μL, 4.04 μmoL, 50 mg/mL) in Tris-8.0 buffer, followed by 520 μM of sortase A* (155 μL, 0.081 μmoL) in 50 mM Tris-Cl pH7.4, 150 mM NaCl. The mixture was shaken at room temperature overnight. LC/MS showed the reaction completed, and that Fc-apelin conjugate was successfully generated. (Sortase A*): Amino acid sequence of Sortase A mutant:

(SEQ ID NO: 16)
MQAKPQIPKDKSKVAGYIEIPDADIKEPVYPGPATREQLNRGVSFAKENQ
SLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVKLE
HHHHHH

where the bold letters represent amino acids which were mutated and the underlined letter represents amino acids described (Chen et al., PNAS, Vol 108, No 28, 2011, 11399-11403) which are not conserved in the original sequence of S aureus sortase A (Mazmanian et al. Science (Washington, D.C.) (1999), 285(5428), 760-763) The sortase A mutant was expressed in E. coli and purified by affinity chromatography exploring the polyhistidine tag comprised at its C-terminus, following established protocols (Carla P. Guimaraes et al.: “Site specific C-terminal and internal loop labeling of proteins using sortase-mediated reactions”, Nature protocols, vol 8, No 9, 2013, 1787-1799).

### 2) Purification and Desalting

The above solution was flowed over a 5 mL HiTrap Mab Select SuRe column (GE Lifesciences #11-0034-95) at 4 mL/min on ATTA XPRESS. Example 21 was washed on the column with 20 column volumes (CV) PBS+0.1% Triton 114 and eluted with 0.1M glycine, pH 2.7, neutralized with 1 M tris-HCl, pH 9 and dialyzed versus PBS. The purified solution was desalted by using Zeba Spin Desalting Column, 5 mL (89891) to give 2 mL target solution, the average concentration was 1.62 mg/mL, and the recoverage was 68%. LCMS (QT2, Protein_20-70 kDa_3 min, AcQuity ProSwift RP-3U 4.6×50 mm, 1.0 mL/min, Eluent A: Water+0.1% Formic Acid, Eluent B: Acetonitrile+0.1% Formic Acid, gradient 2% to 98% B/A over 3 mins): Rₜ=1.55 minutes, MS [M+H] 59346.5000.

After the sortase-mediated conjugation, the resulting amino acid sequence of the Fc-apelin peptide conjugate is as follows:

(SEQ ID NO: 17)
METDTLLLWV LLLWVPGSTG DKTHTCPPCP APEAAGGPSV
FLFPPKPKDT LMISRTPEVT CVVVDVSHED PEVKFNWYVD
GVEVHNAKTK PREEQYNSTY RVVSVLTVLH QDWLNGKEYK
CKVSNKALPA PIEKTISKAK GQPREPQVYT LPPSREEMTK
NQVSLTCLVK GFYPSDIAVE WESNGQPENN YKTTPPVLDS
DGSFFLYSKL TVDKSRWQQG NVFSCSVMHE ALHNHYTQKS
LSLSPGKGGG GSLPETGGGGGQRPRLC*HKGP (Nle)
C*F-COOH (disulfide C¹¹-C¹⁷).

wherein GGGGS (SEQ ID NO: 9) represents the linker, LPETGGGGG (SEQ ID NO: 18) represents the sortase transfer signature, and QRPRLC*HKGP (Nle) C*F—COOH (disulfide C¹¹-C¹⁷) (SEQ ID NO: 19) represents the apelin peptide,

Other sortase mutants, as described herein, can also be used with the same reaction conditions as described in this example to generate a conjugate molecule, e.g., an Fc-apelin conjugate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

1. A sortase molecule, or a purified or isolated preparation thereof, which comprises the amino acid sequence of SEQ ID NO:3, comprising: a mutation selected from Pro94 (P94), Asp160 (D160), Asp165 (D165), Lys190 (K190) and Lys196 (K196); and a mutation selected from Glu105 (E105) and Glu108 (E108); and having at least 90% homology with SEQ ID NO:3.

2. The sortase molecule of claim 1, which comprises the amino acid sequence of SEQ ID NO:3, comprising: a mutation selected from Pro94 (P94), Asp160 (D160), Asp165 (D165), Lys190 (K190) and Lys196 (K196); and a mutation selected from Glu105 (E105) and Glu108 (E108); and otherwise differing from SEQ ID NO:3 by no more than 1, 2, 3, 4, 5, 6, 7, 8, 9, or 10 amino acid residues.

3. The sortase molecule of claim 1, which comprises the amino acid sequence of SEQ ID NO:3, comprising: a mutation selected from Pro94 (P94), Asp160 (D160), Asp165 (D165), Lys190 (K190) and Lys196 (K196); and a mutation selected from Glu105 (E105) and Glu108 (E108).

4. The sortase molecule of claim 1, which comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q).

5. The sortase molecule of claim 1, which comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q); and having at least 90% homology with SEQ ID NO:3.

6. The sortase molecule of claim 1, which comprises the amino acid sequence of SEQ ID NO:3, comprising: a mutation selected from Pro94Arg (P94R), Asp160Asn (D160N), Asp165Ala (D165A), Lys190Glu (K190E) and Lys196Thr (K196T); and a mutation selected from Glu105Lys (E105K) and Glu108Gln (E108Q); and otherwise differing from SEQ ID NO:3 by no more than 1, 2, 3, 4, 5, 6, 7, 8, 9, or 10 amino acid residues.

7. The sortase molecule of claim 1, which comprises the amino acid sequence of SEQ ID NO:3, comprising the following mutations: Pro94 (P94), Glu105 (E105), Glu108 (E108), Asp160 (D160), Asp165 (D165), Lys190 (K190) and Lys196 (K196); and having at least 90% homology with SEQ ID NO:3.

8. The sortase molecule of claim 1, which comprises the amino acid sequence of SEQ ID NO:3, comprising the following mutations: Pro94 (P94), Glu105 (E105), Glu108 (E108), Asp160 (D160), Asp165 (D165), Lys190 (K190) and Lys196 (K196) and otherwise differing from SEQ ID NO:3 by no more than 1, 2, 3, 4, 5, 6, 7, 8, 9, or 10 amino acid residues.

9. The sortase molecule of claim 1, which comprises the amino acid sequence of SEQ ID NO:3, comprising the following mutations: Pro94 (P94), Glu105 (E105), Glu108 (E108), Asp160 (D160), Asp165 (D165), Lys190 (K190), and Lys196 (K196).

10. The sortase molecule of claim 1, which comprises the amino acid sequence of SEQ ID NO:5.

11. A nucleic acid encoding the sortase molecule of claim 1.

12. A vector comprising the nucleic acid of claim 11.

13. A cell comprising the nucleic acid of claim 11.

14. A method of making a sortase molecule, comprising, providing a cell comprising a nucleic acid or vector that comprises sequence that encodes the sortase molecule of claim 1, and recovering the sortase molecule from the cell or secreted by the cell.

15. A method of coupling a first moiety to a second moiety, comprising:
a) providing the first moiety coupled to a sortase acceptor motif and the second moiety coupled to a sortase recognition motif;
b) contacting the first moiety coupled to a sortase acceptor motif with:
(i) a sortase molecule and the second moiety coupled to a sortase recognition motif; or
(ii) a complex comprising the second moiety coupled to a cleaved sortase recognition motif and a sortase molecule;
under conditions sufficient to allow transfer of a second moiety coupled to a cleaved sortase recognition motif to the sortase acceptor motif coupled to the first moiety,
thereby coupling a first moiety to a second moiety,
provided that, the sortase molecule is the sortase molecule of claim 1.

16. A method of providing a cell having a moiety attached thereto, comprising
a) providing a sortase acceptor motif coupled to a first moiety disposed in or on a precursor cell;
b) contacting the precursor cell with
(i) a sortase molecule and a second moiety coupled to a sortase recognition motif; or
(ii) a complex comprising the second moiety coupled to a cleaved sortase recognition motif and a sortase molecule,
under conditions sufficient to allow transfer of a second moiety coupled to a cleaved sortase recognition motif to the sortase acceptor motif coupled to the first moiety,
provided that, the sortase molecule is the sortase molecule of claim 1,
thereby providing cell having a moiety attached thereto.

17. A method of providing a purified preparation of a first moiety coupled to a second moiety, comprising:
providing the first moiety coupled to the second moiety, and
separating the first moiety coupled to the second moiety from a sortase molecule,
thereby providing a purified preparation of a first moiety coupled to a second moiety,
wherein the sortase molecule is the sortase molecule of claim 1.

18. (canceled)


SEQUENCE LISTING (49 sequences)

SEQ ID NO: 1 - protein, 206 aa, Staphylococcus aureus
MKKWTNRLMTIAGVVLILVAAYLFAKPHIDNYLHDKDKDEKIEQYDKNVKEQASKDNKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 2 - protein, 59 aa, Staphylococcus aureus
MKKWTNRLMTIAGVVLILVAAYLFAKPHIDNYLHDKDKDEKIEQYDKNVKEQASKDNKQ

SEQ ID NO: 3 - protein, 147 aa, Staphylococcus aureus
QAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 4 - DNA, 621 bp, Staphylococcus aureus
atgaaaaaatggacaaatcgattaatgacaatcgctggtgtagtacttatcctagtggcagcatatttgtttgctaaaccacatatcgataattatcttcacgataaagataaagatgaaaagattgaacaatatgataaaaatgtaaaagaacaggcgagtaaagacaataagcagcaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaataa

SEQ ID NO: 5 - protein, 156 aa, Artificial Sequence
  source: /note=“Description of Artificial Sequence Synthetic polypeptide”
MQAKPQIPKDKSKVAGYIEIPDADIKEPVYPGPATREQLNRGVSFAKENQSLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 6 - protein, 284 aa, Artificial Sequence
  source: /note=“Description of Artificial Sequence Synthetic polypeptide”
METDTLLLWVLLLWVPGSTGEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSLPETGGLDVLFEGPHHHHHHHH

SEQ ID NO: 7 - protein, 4 aa, Artificial Sequence
  source: /note=“Description of Artificial Sequence Synthetic peptide”
GGGK

SEQ ID NO: 8 - DNA, 817 bp, Artificial Sequence
  source: /note=“Description of Artificial Sequence Synthetic polynucleotide”
gctagccaccatggaaaccgacaccctgctgctgtgggtgctgctgctgtgggtgccaggcagcaccggcgataagacccacacctgtcctccctgtcctgcccctgaagctgctggcggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccagcccccatcgagaaaaccatcagcaaggccaagggccagccccgcgaaccccaggtgtacacactgccccctagccgggaagagatgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtccctgagcctgagccctggaaaaggcggcggaggctctctgcctgaaacaggcggactggaagtgctgttccagggcccctaagaattc

SEQ ID NO: 9 - protein, 5 aa, Artificial Sequence
  source: /note=“Description of Artificial Sequence Synthetic peptide”
GGGGS

SEQ ID NO: 10 - protein, 14 aa, Artificial Sequence
  source: /note=“Description of Artificial Sequence Synthetic peptide”
LPETGGLEVLFQGP

SEQ ID NO: 11 - protein, 10 aa, Artificial Sequence
  source: /note=“Description of Artificial Sequence Synthetic peptide”
GGLEVLFQGP

SEQ ID NO: 12 - protein, 266 aa, Artificial Sequence
  source: /note=“Description of Artificial Sequence Synthetic polypeptide”
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLPETGGLEVLFQGP

SEQ ID NO: 13 - protein, 16 aa, Artificial Sequence
  source: /note=“Description of Artificial Sequence Synthetic peptide”
GGGGGQRPCLSCKGPX

SEQ ID NO: 14 - protein, 272 aa, Artificial Sequence
  source: /note=“Description of Artificial Sequence Synthetic polypeptide”
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLPETGGGGGQRPCLSCKGPX

SEQ ID NO: 15 - protein, 18 aa, Artificial Sequence
  source: /note=“Description of Artificial Sequence Synthetic peptide”
GGGGGQRPRLCHKGPXCF

SEQ ID NO: 16 - protein, 156 aa, Artificial Sequence; identical to SEQ ID NO: 5
  source: /note=“Description of Artificial Sequence Synthetic polypeptide”

SEQ ID NO: 17 - protein, 274 aa, Artificial Sequence
  source: /note=“Description of Artificial Sequence Synthetic polypeptide”
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLPETGGGGGQRPRLCHKGPXCF

SEQ ID NO: 18 - protein, 9 aa, Artificial Sequence
  source: /note=“Description of Artificial Sequence Synthetic peptide”
LPETGGGGG

SEQ ID NO: 19 - protein, 13 aa, Artificial Sequence
  source: /note=“Description of Artificial Sequence Synthetic peptide”
QRPRLCHKGPXCF

SEQ ID NO: 20 - protein, 19 aa, Artificial Sequence
  source: /note=“Description of Artificial Sequence Synthetic peptide”
GGGGSLPETGGLEVLFQGP

SEQ ID NO: 21 - protein, 16 aa, Artificial Sequence
  source: /note=“Description of Artificial Sequence Synthetic peptide”
GGGGGQRPCLSCKGPX

SEQ ID NO: 22 - protein, 25 aa, Artificial Sequence
  source: /note=“Description of Artificial Sequence Synthetic peptide”
GGGGSLPETGGGGGQRPCLSCKGPX

SEQ ID NO: 23 - protein, 16 aa, Artificial Sequence
  source: /note=“Description of Artificial Sequence Synthetic peptide”
GGGGGQRPCLSCKGPX

SEQ ID NO: 24 - protein, 16 aa, Artificial Sequence
  source: /note=“Description of Artificial Sequence Synthetic peptide”
GGGGGQRPCLSCKGPX

SEQ ID NO: 25 - protein, 16 aa, Artificial Sequence
  source: /note=“Description of Artificial Sequence Synthetic peptide”
GGGGGQRPCLSCKGPX

SEQ ID NO: 26 - protein, 19 aa, Artificial Sequence
  source: /note=“Description of Artificial Sequence Synthetic peptide”
GGGGSLPETGGLEVLFQGP

SEQ ID NO: 27 - protein, 18 aa, Artificial Sequence
  source: /note=“Description of Artificial Sequence Synthetic peptide”
GGGGGQRPRLCHKGPXCF

SEQ ID NO: 28 - protein, 27 aa, Artificial Sequence
  source: /note=“Description of Artificial Sequence Synthetic peptide”
GGGGSLPETGGGGGQRPRLCHKGPXCF

SEQ ID NO: 29 - protein, 18 aa, Artificial Sequence
  source: /note=“Description of Artificial Sequence Synthetic peptide”
GGGGGQRPRLCHKGPXCF

SEQ ID NO: 30 - protein, 18 aa, Artificial Sequence
  source: /note=“Description of Artificial Sequence Synthetic peptide”
GGGGGQRPRLCHKGPXCF

SEQ ID NO: 31 - protein, 18 aa, Artificial Sequence
  source: /note=“Description of Artificial Sequence Synthetic peptide”
GGGGGQRPRLCHKGPXCF

SEQ ID NO: 32 - protein, 6 aa, Artificial Sequence
  source: /note=“Description of Artificial Sequence Synthetic 6xHis tag”
HHHHHH

SEQ ID NO: 33 - protein, 8 aa, Artificial Sequence
  source: /note=“Description of Artificial Sequence Synthetic 8xHis tag”
HHHHHHHH

SEQ ID NO: 34 - protein, 4 aa, Artificial Sequence
  source: /note=“Description of Artificial Sequence Synthetic peptide”
GGGG

SEQ ID NO: 35 - protein, 5 aa, Artificial Sequence
  source: /note=“Description of Artificial Sequence Synthetic peptide”
GGGGG

SEQ ID NO: 36 - protein, 4 aa, Artificial Sequence
  source: /note=“Description of Artificial Sequence Synthetic peptide”
AAAA

SEQ ID NO: 37 - protein, 5 aa, Artificial Sequence
  source: /note=“Description of Artificial Sequence Synthetic peptide”
AAAAA

SEQ ID NO: 38 - protein, 5 aa, Artificial Sequence
  source: /note=“Description of Artificial Sequence Synthetic peptide”
LPXTG

SEQ ID NO: 39 - protein, 5 aa, Artificial Sequence
  source: /note=“Description of Artificial Sequence Synthetic peptide”
LPETG

SEQ ID NO: 40 - protein, 6 aa, Artificial Sequence
  source: /note=“Description of Artificial Sequence Synthetic peptide”
GGGGGG

SEQ ID NO: 41 - protein, 6 aa, Artificial Sequence
  source: /note=“Description of Artificial Sequence Synthetic peptide”
AAAAAA

SEQ ID NO: 42 - protein, 6 aa, Artificial Sequence
  source: /note=“Description of Artificial Sequence Synthetic peptide”
LPXTGG

SEQ ID NO: 43 - protein, 4 aa, Artificial Sequence
  source: /note=“Description of Artificial Sequence Synthetic peptide”
GGGS

SEQ ID NO: 44 - protein, 20 aa, Artificial Sequence
  source: /note=“Description of Artificial Sequence Synthetic peptide”
GGGGSGGGGSGGGGSGGGGS

SEQ ID NO: 45 - protein, 15 aa, Artificial Sequence
  source: /note=“Description of Artificial Sequence Synthetic peptide”
GGGGSGGGGSGGGGS

SEQ ID NO: 46 - protein, 6 aa, Artificial Sequence
  source: /note=“Description of Artificial Sequence Synthetic peptide”
LPETGG

SEQ ID NO: 47 - protein, 21 aa, Artificial Sequence
  source: /note=“Description of Artificial Sequence Synthetic peptide”
LSLSPGKGGGGSLPETGGGGG

SEQ ID NO: 48 - protein, 11 aa, Artificial Sequence
  source: /note=“Description of Artificial Sequence Synthetic peptide”
QRPCLSCKGPX

SEQ ID NO: 49 - protein, 8 aa, Artificial Sequence
  source: /note=“Description of Artificial Sequence Synthetic peptide”
LPETGGGK